(19) United States
(12) Patent Application Publication
(10) Pub. No.: US 2026/0294317 A1
(43) Pub. Date: Oct. 1, 2026

(54) SYSTEMS AND METHODS FOR IDENTIFYING HYPERACUTE T-WAVES

(71) Applicant: POWERFUL MEDICAL s.r.o., New York, NY (US)

(72) Inventors: Harvey Pendell MEYERS, Charlotte, NC (US); František SIMANCÍK, Bratislava (SK); Robert HERMAN, Bratislava (SK); Adam RAFAJDUS, Bratislava (SK); Stephen W. Smith, Minneapolis, MN (US)

(21) Appl. No.: 19/633,929

(22) Filed: Mar. 30, 2026

Related U.S. Application Data
(60) Provisional application No. 63/779,955, filed on Mar. 28, 2025.

(51) Int. Cl. A61B 5/355; A61B 5/00

(52) U.S. Cl. CPC A61B 5/355; A61B 5/746

(57) ABSTRACT

Systems and methods for identifying hyperacute T-waves in electrocardiogram (ECG) data are provided. ECG signal data from at least one lead is received, and T-wave and QRS complex components are identified. A T-wave magnitude metric quantifying relative T-wave size and a T-wave symmetry metric quantifying temporal distribution of a T-wave peak are computed. A hyperacute T-wave score is generated based on the magnitude metric and the symmetry metric, and an output indicating presence or absence of a hyperacute T-wave is produced. ECG data from multiple leads may be evaluated to determine whether a threshold condition is satisfied across contiguous leads. Further disclosed are systems and methods for quantifying T-wave morphology by extracting a first metric characterizing relative T-wave size and a second metric characterizing temporal asymmetry or symmetry, computing a composite morphology score, and classifying the T-wave based on the composite morphology score.

### CROSS-REFERENCE TO RELATED APPLICATIONS

This application claims the benefit of U.S. Provisional Patent Application No. 63/779,955, filed Mar. 28, 2025, which is incorporated by reference herein in its entirety.

### FIELD OF THE INVENTION

The present application relates to systems and methods for identifying hyperacute T-waves in electrocardiogram data. More specifically, the present disclosure relates to computer-implemented methods for quantifying T-wave morphology using magnitude, symmetry, and optionally additional morphological metrics to produce a hyperacute T-wave score, to systems and clinical workflows that integrate such scoring into emergency department decision-making pathways for detection of occlusion myocardial infarction, and to methods for training classification models and generating objective ground-truth labels for hyperacute T-wave identification.

### BACKGROUND

Hyperacute T waves (HATWs) may provide an electrocardiographic (ECG) indicator associated with acute coronary occlusion myocardial infarction (OMI). Clinical guidance has recognized HATWs as potentially relevant in identifying presentations that may warrant urgent reperfusion evaluation. However, HATW identification has generally remained dependent on subjective visual assessment, and conventional ECG analysis workflows have lacked a consistent objective framework for quantitatively characterizing HATW morphology in a manner suitable for automated computation.

Some prior approaches have attempted to characterize HATW-related morphology using amplitude-based measurements, such as T-wave amplitude alone or ratios involving T-wave amplitude and QRS amplitude. While such approaches may capture certain waveform characteristics, they do not adequately distinguish pathological hyperacute morphology from benign high-amplitude T waves across clinically relevant ECG presentations, and they have not provided a sufficiently robust basis for software-driven identification of HATWs in the context of acute coronary occlusion assessment.

Accordingly, improved systems and methods are needed for processing ECG data to identify waveform characteristics associated with hyperacute T waves using objective, quantifiable morphology measures. Such systems and methods may preferably distinguish pathological HATWs from non-pathological T-wave morphologies, support reproducible processor-executed analysis, and generate outputs suitable for incorporation into automated ECG interpretation, decision support, and related clinical workflows.

### SUMMARY

In some aspects, the techniques described herein relate to a computer-implemented method for identifying hyperacute T-waves in electrocardiogram (ECG) data, the method including: (a) receiving, by at least one processor, ECG signal data representing cardiac electrical activity of a subject, the ECG signal data including waveform data from at least one ECG lead; (b) identifying, by the at least one processor, at least one T-wave component and at least one QRS complex component within the ECG signal data for the at least one ECG lead; (c) computing, by the at least one processor, a T-wave magnitude metric for the at least one ECG lead, wherein the T-wave magnitude metric quantifies a size of the T-wave component relative to a size of the QRS complex component; (d) computing, by the at least one processor, a T-wave symmetry metric for the at least one ECG lead, wherein the T-wave symmetry metric quantifies a temporal distribution of a peak of the T-wave component within a duration of the T-wave component; (e) generating, by the at least one processor, a hyperacute T-wave (HATW) score for the at least one ECG lead based at least in part on the T-wave magnitude metric and the T-wave symmetry metric; and (f) producing, by the at least one processor, an output based at least in part on the HATW score, the output being indicative of a presence or absence of a hyperacute T-wave in the at least one ECG lead.

In some aspects, the techniques described herein relate to a method, wherein computing the T-wave magnitude metric includes computing a ratio of an area under the T-wave component to an amplitude of the QRS complex component, wherein the area under the T-wave component is measured from a J-point to an end of the T-wave, and wherein the amplitude of the QRS complex component is measured as a difference between a maximum value and a minimum value of the QRS complex.

In some aspects, the techniques described herein relate to a method, wherein computing the T-wave symmetry metric includes computing a ratio of (i) a time interval from a peak of the T-wave component to an end of the T-wave component, to (ii) a time interval from a J-point to the peak of the T-wave component.

In some aspects, the techniques described herein relate to a method, wherein generating the HATW score includes applying a trained classification model to at least the T-wave magnitude metric and the T-wave symmetry metric, the trained classification model having been trained on expert-annotated ECG data including leads labeled as hyperacute or non-hyperacute by human ECG experts.

In some aspects, the techniques described herein relate to a method, wherein: the ECG signal data includes waveform data from a plurality of ECG leads; the method includes computing the T-wave magnitude metric, the T-wave symmetry metric, and the HATW score for each lead of the plurality of ECG leads; and producing the output includes determining that at least two contiguous leads of the plurality of ECG leads each have a HATW score satisfying a predetermined threshold condition.

In some aspects, the techniques described herein relate to a method, wherein the trained classification model includes a logistic regression model or a feedforward neural network including at least two hidden units with logistic activation functions.

In some aspects, the techniques described herein relate to a method, wherein the HATW score is computed using lead-group-specific model parameters, wherein the plurality of ECG leads are divided into at least two lead groups, and wherein each lead group has distinct model parameters optimized for morphological characteristics of that lead group.

In some aspects, the techniques described herein relate to a method, further including, prior to computing the T-wave magnitude metric and the T-wave symmetry metric, determining that the at least one ECG lead satisfies one or more eligibility criteria, the one or more eligibility criteria including at least one of: (i) a QRS complex duration not exceeding a predetermined duration threshold; (ii) a total QRS complex amplitude meeting or exceeding a predetermined QRS amplitude threshold; (iii) a positive T-wave amplitude meeting or exceeding a predetermined T-wave amplitude threshold; (iv) a T-wave onset-to-peak duration and a T-wave peak-to-end duration each meeting or exceeding a predetermined minimum T-wave timing threshold; and (v) the T-wave component being neither inverted nor biphasic according to predetermined morphological criteria.

In some aspects, the techniques described herein relate to a method, wherein the predetermined threshold condition includes a mean HATW score of the at least two contiguous leads meeting or exceeding a threshold value.

In some aspects, the techniques described herein relate to a method, wherein the threshold value is approximately 0.7.

In some aspects, the techniques described herein relate to a method, wherein generating the HATW score further includes computing an ST-segment depression metric for the at least one ECG lead, and wherein the HATW score is based at least in part on the T-wave magnitude metric, the T-wave symmetry metric, and the ST-segment depression metric.

In some aspects, the techniques described herein relate to a method, wherein the logistic regression model or feedforward neural network includes: a magnitude sub-score computed by applying a first logistic function to at least the area under the T-wave component and the amplitude of the QRS complex component; a symmetry sub-score computed by applying a second logistic function to at least a T-wave peak-to-end time and a T-wave onset-to-peak time; and the HATW score computed by applying a third logistic function to at least the magnitude sub-score and the symmetry sub-score.

In some aspects, the techniques described herein relate to a method, wherein the HATW score is computed according to lead-group-specific equations, wherein each equation includes a logistic function of a weighted combination of a bias term, a scaled magnitude sub-score, and a scaled symmetry sub-score, with lead-group-specific weights and bias values.

In some aspects, the techniques described herein relate to a method, wherein the output is indicative of a likelihood of occlusion myocardial infarction (OMI) in the subject.

In some aspects, the techniques described herein relate to a method, wherein producing the output includes generating a clinical alert recommending at least one of: emergent cardiac catheterization, emergent reperfusion therapy, or expedited cardiology consultation.

In some aspects, the techniques described herein relate to a method, further including constructing, by the at least one processor, at least one median beat representation from the ECG signal data prior to identifying the T-wave component and the QRS complex component.

In some aspects, the techniques described herein relate to a system for identifying hyperacute T-waves in electrocardiogram (ECG) data, the system including: at least one processor; and at least one memory storing instructions that, when executed by the at least one processor, cause the system to: (a) receive ECG signal data representing cardiac electrical activity of a subject, the ECG signal data including waveform data from at least one ECG lead; (b) identify at least one T-wave component and at least one QRS complex component within the ECG signal data for the at least one ECG lead; (c) compute a T-wave magnitude metric for the at least one ECG lead, wherein the T-wave magnitude metric quantifies a size of the T-wave component relative to a size of the QRS complex component; (d) compute a T-wave symmetry metric for the at least one ECG lead, wherein the T-wave symmetry metric quantifies a temporal distribution of a peak of the T-wave component within a duration of the T-wave component; (e) generate a hyperacute T-wave (HATW) score for the at least one ECG lead based at least in part on the T-wave magnitude metric and the T-wave symmetry metric; and (f) produce an output based at least in part on the HATW score, the output being indicative of a presence or absence of a hyperacute T-wave in the at least one ECG lead.

In some aspects, the techniques described herein relate to a system, wherein: the ECG signal data includes waveform data from a plurality of ECG leads; the instructions further cause the system to compute the T-wave magnitude metric, the T-wave symmetry metric, and the HATW score for each lead of the plurality of ECG leads using lead-group-specific model parameters; the instructions further cause the system to determine that at least two contiguous leads of the plurality of ECG leads satisfy a predetermined threshold condition based on a mean HATW score of the at least two contiguous leads; and the output includes a clinical alert indicative of a likelihood of occlusion myocardial infarction (OMI) and recommending at least one of emergent cardiac catheterization, emergent reperfusion therapy, or expedited cardiology consultation.

In some aspects, the techniques described herein relate to a non-transitory computer-readable medium storing instructions that, when executed by at least one processor, cause the at least one processor to perform operations including: (a) receiving ECG signal data representing cardiac electrical activity of a subject, the ECG signal data including waveform data from at least one ECG lead; (b) identifying at least one T-wave component and at least one QRS complex component within the ECG signal data for the at least one ECG lead; (c) computing a T-wave magnitude metric for the at least one ECG lead, wherein the T-wave magnitude metric quantifies a size of the T-wave component relative to a size of the QRS complex component; (d) computing a T-wave symmetry metric for the at least one ECG lead, wherein the T-wave symmetry metric quantifies a temporal distribution of a peak of the T-wave component within a duration of the T-wave component; (e) generating a hyperacute T-wave (HATW) score for the at least one ECG lead based at least in part on the T-wave magnitude metric and the T-wave symmetry metric; and (f) producing an output based at least in part on the HATW score, the output being indicative of a presence or absence of a hyperacute T-wave in the at least one ECG lead.

In some aspects, the techniques described herein relate to a non-transitory computer-readable medium, wherein the operations further include: constructing at least one median beat representation from the ECG signal data prior to identifying the at least one T-wave component and the at least one QRS complex component; applying a trained classification model to at least the T-wave magnitude metric and the T-wave symmetry metric, the trained classification model having been trained on expert-annotated ECG data including leads labeled as hyperacute or non-hyperacute by human ECG experts; and generating the HATW score further based at least in part on an ST-segment depression metric for the at least one ECG lead.

In some aspects, the techniques described herein relate to a computer-implemented method for quantifying T-wave morphology in electrocardiogram (ECG) data, the method including: (a) receiving, by at least one processor, ECG signal data including waveform data from at least one ECG lead; (b) extracting, by the at least one processor, from the ECG signal data, at least: (i) a first metric characterizing a relative size of a T-wave with respect to a QRS complex in the at least one ECG lead, and (ii) a second metric characterizing a temporal asymmetry or symmetry of the T-wave in the at least one ECG lead; (c) computing, by the at least one processor, a composite morphology score from at least the first metric and the second metric; and (d) classifying, by the at least one processor, the T-wave in the at least one ECG lead as hyperacute or non-hyperacute based at least in part on the composite morphology score.

### BRIEF DESCRIPTION OF THE FIGURES

The accompanying drawings, which are incorporated herein and form part of the specification, illustrate embodiments of the present disclosure and, together with the description, further serve to explain the principles and to enable a person skilled in the pertinent art to make and use the embodiments.

The drawings are not necessarily to scale. Where appropriate, similar or identical reference numbers are used to refer to similar or identical components.

FIG. 1 is a diagram illustrating the computation of a HATW score, including magnitude score computation, symmetry score computation, and combined HATW score, together with examples of hyperacute and non-hyperacute T-wave morphologies.

FIG. 2 includes scatter plots comparing T-wave magnitude and symmetry scores for control T-waves versus expert-annotated hyperacute T-waves across three lead groups, together with lead-group-specific HATW score equations including all model coefficients.

FIG. 3 shows clinical example ECGs: (A) a true OMI case with HATWs identified by the HATW score, and (B) a non-OMI case with ST elevation but without HATWs as determined by the HATW score.

FIG. 4 shows median beat representations of the ECGs from FIG. 3, displaying per-lead HATW score, magnitude score, and symmetry score with visual differentiation between leads above and below threshold.

FIG. 5 is a flowchart depicting clinical outcomes when the HATW score is applied as a STEMI equivalent finding in a validation cohort.

FIG. 6 is a block diagram of a computing system on which the described systems and methods may be executed.

FIG. 7 is an example ECG with expert annotations marking hyperacute T-waves in specific leads.

FIG. 8 is a heatmap showing interrater reliability between two ECG experts for hyperacute T-wave annotation.

FIG. 9 includes scatter plots of per-lead scores for example cases superimposed on lead-group-specific decision boundaries.

FIG. 10 is a block diagram of a system for identifying hyperacute T-waves.

FIG. 11 is a flowchart illustrating a method for identifying hyperacute T-waves in ECG data.

FIG. 12 is a flowchart illustrating a method for training a hyperacute T-wave classification model.

FIG. 13 is a flowchart illustrating a clinical workflow method for integrating HATW detection into emergency department reperfusion decision-making.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION

In the following description, for the purposes of explanation, numerous specific details are set forth in order to provide a thorough understanding of the various embodiments. It will be appreciated, however, by those having skill in the art, that the embodiments may be practiced without these specific details, or with an equivalent arrangement. In other cases, well-known structures and devices are shown in block diagram form in order to avoid unnecessarily obscuring the embodiments.

To mitigate the problems described herein, the inventors had to both invent solutions and, in some cases just as importantly, recognize problems overlooked (or not yet foreseen) by others in the field of automating event prediction and missed event detection. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

The present disclosure relates to computer-implemented systems and methods for identifying hyperacute T-wave morphology in electrocardiogram (ECG) data and for using such identification in automated analysis, clinical decision support, model training, visualization, and downstream diagnostic workflows. In various embodiments, one or more processors receive ECG waveform data from one or more leads, identify waveform components including QRS complexes and T-wave portions, compute one or more morphology metrics characterizing the shape of the T-wave relative to the surrounding waveform, and generate a score, classification, or other output indicative of whether the ECG data exhibits hyperacute T-wave morphology. The disclosed techniques may be implemented on local acquisition devices, on remote servers, in distributed processing environments, or in combinations thereof, and may operate on 12-lead ECG data, reduced-lead ECG data, median beat representations, serial ECG recordings, or other waveform-derived data structures.

In an exemplary embodiment, the disclosed techniques quantify hyperacute T-wave morphology using at least two morphology metrics. A first morphology metric may characterize a relative size or bulk of a T-wave with respect to a QRS complex in a given ECG lead, and may be referred to herein as a T-wave magnitude metric. A second morphology metric may characterize temporal symmetry or asymmetry of the T-wave, and may be referred to herein as a T-wave symmetry metric. One or more processors may compute these metrics from delineated waveform components and may combine them to produce a hyperacute T-wave (HATW) score. In some embodiments, the HATW score is a continuous value, such as a value on a bounded scale, and in some embodiments the HATW score is converted into a binary, categorical, or thresholded output indicating whether the corresponding T-wave or ECG satisfies one or more hyperacute criteria.

Although magnitude and symmetry are described extensively herein as exemplary metrics, the present disclosure is not limited to those two metrics or to any single mathematical formulation thereof. In some embodiments, the score generation process may further incorporate one or more additional morphology metrics, such as an ST-segment depression metric, a convexity metric, an inter-lead relationship metric, a spatial gradient metric, a reciprocal-lead metric, or other quantitative representations of T-wave morphology. In some embodiments, the score may be generated from any subset of the disclosed metrics. In some embodiments, the score may be generated using explicitly computed features, while in other embodiments a trained model may learn internal representations from waveform data that correspond, directly or indirectly, to the morphology concepts described herein. Accordingly, the disclosed HATW score may be understood both as an exemplary scoring implementation based on concrete magnitude and symmetry measures and, more broadly, as an instance of a processor-executed composite morphology score derived from one or more quantitative characterizations of T-wave morphology.

In some embodiments, the disclosed techniques operate on a per-lead basis and additionally perform ECG-level evaluation by combining lead-level outputs according to one or more threshold conditions. For example, a processor may generate a score for each eligible lead, determine whether one or more contiguous leads satisfy a threshold condition, and produce an ECG-level output indicating whether hyperacute morphology is present. In some embodiments, lead-group-specific models or parameter sets are used for different anatomical lead groups. In some embodiments, lead eligibility rules are applied before score generation to reduce instability associated with ineligible or poorly defined waveform segments. In some embodiments, preprocessing may include beat selection, median beat construction, waveform segmentation, baseline normalization, noise attenuation, or artifact handling to improve reproducibility and runtime robustness.

The output generated by the disclosed techniques may take various forms depending on the deployment context. In some embodiments, the output may comprise a per-lead HATW score, a per-ECG determination, a confidence indicator, an occlusion myocardial infarction (OMI) likelihood indicator, a STEMI-equivalent indication, or a clinical alert recommending one or more actions. In some embodiments, the output may be presented visually in association with ECG waveforms, including overlay displays, color-coded threshold indications, per-lead component-score displays, heatmaps, or other graphical arrangements. In some embodiments, the output may be transmitted to clinical information systems, diagnostic workstations, bedside monitors, mobile devices, electronic health record systems, or other computing platforms. In some embodiments, outputs from one ECG may be compared with outputs from a subsequent ECG to generate temporal comparison data, trend alerts, or other serial-analysis outputs.

In some embodiments, the disclosed techniques further include training and model-generation workflows. For example, one or more processors may receive ECG recordings and expert annotations, compute morphology metrics for annotated leads, apply inclusion or exclusion criteria to prepare a training data set, and train one or more classification models that output HATW scores or related morphology indicators. In some embodiments, positive training instances are weighted according to severity annotations. In some embodiments, separate models are trained for separate lead groups. In some embodiments, HATW scores or thresholded HATW labels generated using the disclosed techniques are used as training targets for downstream cardiac diagnostic models, including models that operate directly on raw waveform data at inference time. Thus, the disclosure encompasses not only runtime HATW identification but also model derivation, calibration, deployment, and use of HATW scoring as an objective supervisory signal.

In some embodiments, the disclosed techniques are integrated into a clinical workflow associated with evaluation of patients presenting with suspected acute coronary syndrome. A processor may evaluate whether ECG data satisfies STEMI criteria, and when STEMI criteria are absent or not satisfied, may apply the HATW analysis described herein to determine whether the ECG exhibits hyperacute morphology that should be treated as a STEMI-equivalent or otherwise clinically actionable finding. In some embodiments, the resulting output may trigger a clinical alert, recommend expedited cardiology review, emergent reperfusion evaluation, or catheterization workflow, provide a suspected culprit artery indication, or support serial monitoring as additional ECG data becomes available. The disclosed techniques may therefore function as a technical ECG-analysis mechanism, a decision-support mechanism, a visualization mechanism, a model-training mechanism, or a component within a broader diagnostic pipeline.

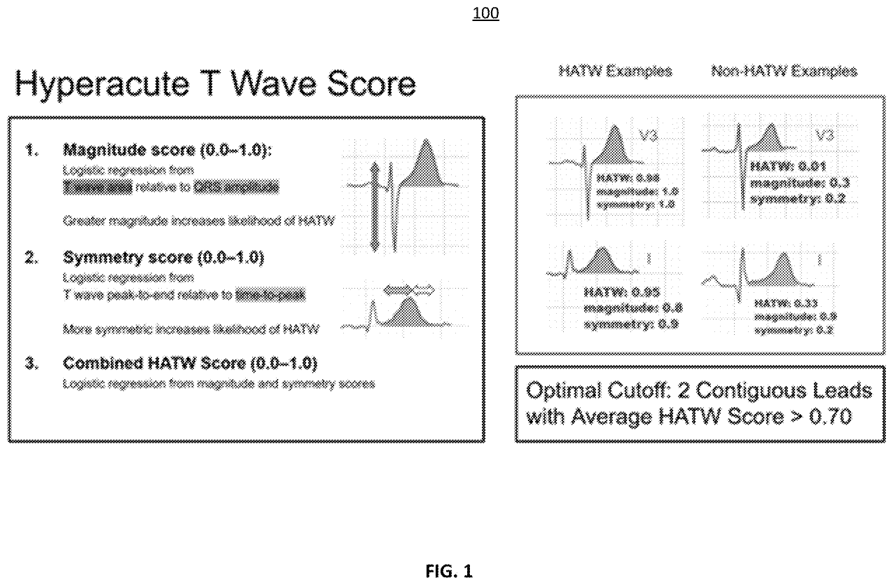

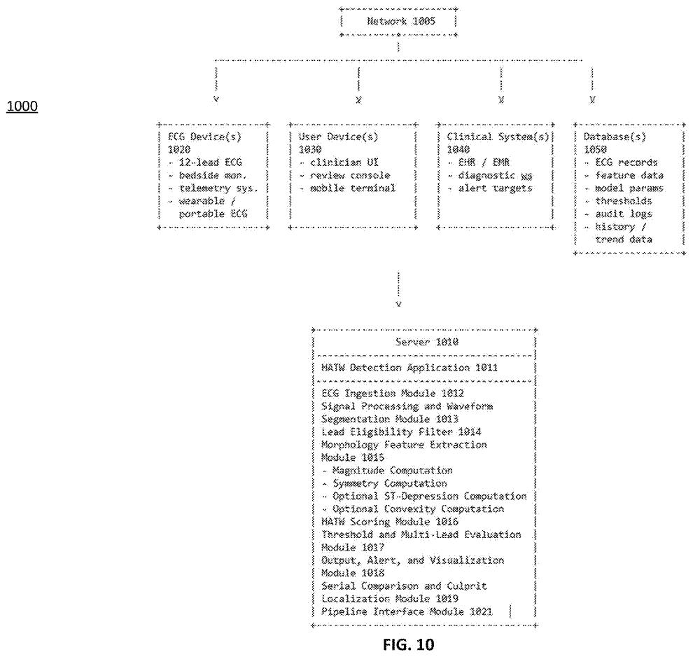

FIGS. 1-9 illustrate exemplary score construction, derivation, validation, annotation, and example-case material associated with the disclosed HATW framework, including morphology definitions, score plots, example ECGs, median beat views, validation-flow material, expert annotation, interrater agreement, and decision-boundary visualizations. These figures reflect exemplary embodiments and experimental support associated with various embodiments and are useful for understanding representative implementations of the disclosed metrics and scoring framework. FIGS. 10-13 illustrate additional architecture and workflow embodiments, including a system architecture for implementing the disclosed techniques, a processor-executed method for identifying hyperacute T-waves, a training workflow for generating one or more HATW models, and a clinical workflow for integrating HATW detection into decision support and alerting pathways. The figures are discussed in turn below.

FIG. 1 illustrates an example arrangement for computing a hyperacute T-wave (HATW) score from ECG waveform morphology and for visually contrasting waveform characteristics associated with hyperacute and non-hyperacute T waves. In various embodiments, FIG. 1 may be understood as depicting a processor-executed scoring framework in which one or more waveform-derived morphology metrics are computed from ECG signal data and combined to generate a score indicative of whether a given T wave exhibits hyperacute morphology. In the exemplary embodiment reflected in FIG. 1, the score generation process includes at least a magnitude score computation, a symmetry score computation, and a combined HATW score computation. FIG. 1 also includes representative examples of T-wave morphologies that the scoring framework characterizes as more consistent with hyperacute morphology and morphologies that the scoring framework characterizes as more consistent with non-hyperacute or baseline T-wave appearance.

In some embodiments, the magnitude-related portion of FIG. 1 illustrates computation of a T-wave magnitude metric from a delineated portion of the ECG waveform corresponding to the T wave and a delineated portion corresponding to the QRS complex. In this exemplary implementation, the T-wave magnitude metric is based on the area under the T wave, measured from the J-point to the end of the T wave, relative to the amplitude of the QRS complex measured from a maximum point to a minimum point of the QRS complex. This formulation captures the relative size or “bulk” of the T wave with respect to the QRS complex and provides a more informative quantitative characterization than isolated T-wave amplitude alone. In some embodiments, the processor may determine the relevant delineation points using waveform segmentation logic, store the resulting boundary positions in memory, numerically integrate or otherwise estimate the T-wave area over the identified interval, compute the QRS amplitude from the identified extrema, and generate a magnitude score or magnitude metric from those values.

In some embodiments, the symmetry-related portion of FIG. 1 illustrates computation of a T-wave symmetry metric from temporal positions within the T-wave portion of the ECG waveform. In some embodiments, the T-wave symmetry metric is based on the time from the T-wave peak to the end of the T wave relative to the time from the J-point or T-wave onset region to the T-wave peak. This metric quantifies the temporal position of the T-wave peak within the overall T-wave duration and provides a measure of symmetry or asymmetry of the waveform shape. In various embodiments, T waves associated with hyperacute morphology tend to exhibit greater symmetry than ordinary non-hyperacute T waves, whose upstroke and downstroke relationships are typically more asymmetric. In some embodiments, the processor may identify the relevant onset, peak, and end locations, compute one or more interval lengths, normalize the intervals by ratio or other transformation, and generate a symmetry score or symmetry metric representing the temporal morphology of the T wave.

In various embodiments, FIG. 1 further illustrates that the magnitude-related metric and symmetry-related metric may be combined to produce a HATW score. In the exemplary embodiment, the magnitude score and symmetry score are each produced using a logistic-regression-based transformation and then combined using an additional logistic-regression stage to produce the HATW score. However, FIG. 1 should not be understood as limited to any single mathematical implementation. Rather, FIG. 1 may be understood more broadly as illustrating that multiple morphology measures derived from the ECG waveform may be processed by one or more score-generation operations to produce a composite output indicative of hyperacute T-wave morphology. In some embodiments, the score may be a continuous numerical value. In some embodiments, the score may be further evaluated against one or more thresholds or decision boundaries to generate a binary or categorical output.

The example waveforms in FIG. 1 help illustrate the morphological distinctions captured by the disclosed scoring approach. In various embodiments, hyperacute T waves are characterized not merely by large peak amplitude, but by a combination of greater relative area and greater symmetry, while non-hyperacute T waves may have lower relative area, greater asymmetry, or both. Thus, FIG. 1 visually reinforces the recognition that simple amplitude measurements alone do not adequately capture the morphology of interest and that a more robust processor-executed characterization may be obtained by jointly evaluating relative T-wave size and temporal symmetry. The examples shown in FIG. 1 are illustrative only and are not intended to limit the disclosed techniques to any specific waveform appearance, polarity convention, or lead type.

In some embodiments, FIG. 1 may also be understood as supporting broader implementations in which the specific magnitude and symmetry formulations shown are exemplary instances of a more general morphology-scoring framework. For example, the relative-size characterization may be implemented using another quantitative measure of T-wave size relative to the QRS complex, and the temporal-symmetry characterization may be implemented using another quantitative measure of the temporal distribution of the T-wave peak or waveform shape. Likewise, the combined score generation may be implemented using logistic regression, another machine learning model, a rule-based scoring function, or other processor-executed logic. Accordingly, while FIG. 1 provides a concrete illustrative embodiment of HATW score computation, the present disclosure is not limited to only the precise formulas or transformations shown in that figure.

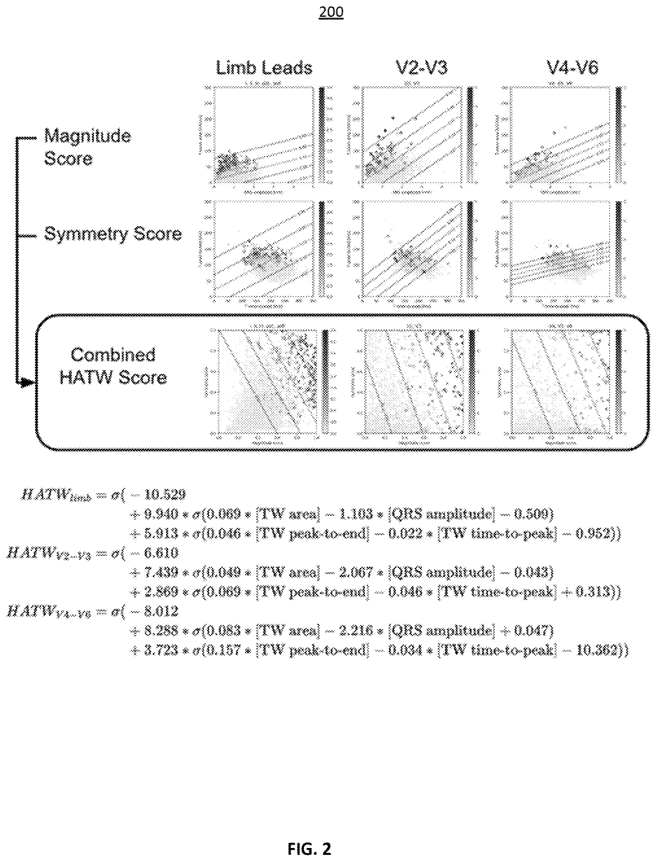

FIG. 2 illustrates example score-distribution and score-generation information associated with the HATW framework across multiple ECG lead groups. In the exemplary embodiment, FIG. 2 includes scatter plots comparing T-wave magnitude and symmetry values, or corresponding magnitude and symmetry scores, for control T waves and expert-annotated hyperacute T waves across a plurality of lead groups. In various embodiments, the lead groups may include limb leads, right precordial leads, and left precordial leads, with separate model behavior or parameters for each group. FIG. 2 further includes lead-group-specific HATW score equations that define how the magnitude-related and symmetry-related information is combined to generate a HATW score for a lead within a given group.

The scatter plots in FIG. 2 illustrate that, in the exemplary data set, expert-annotated hyperacute T waves tend to occupy a different region of the magnitude-symmetry space than control T waves. More particularly, hyperacute T waves generally demonstrate greater T-wave magnitude and greater T-wave symmetry than control T waves, and FIG. 2 provides a visual representation of that separation. In some embodiments, the plotted points may be colored, shaded, or otherwise differentiated according to control status, hyperacute status, or degree of expert-assigned severity. In this manner, FIG. 2 demonstrates that the disclosed morphology metrics are not merely theoretical descriptors, but may be used as processor-computed quantities that separate hyperacute and non-hyperacute waveforms in a structured feature space.

FIG. 2 also illustrates that score generation may be parameterized differently for different lead groups. In the exemplary implementation, separate lead-group-specific equations or coefficient sets are used to account for systematic morphological differences among different categories of ECG leads. Thus, a processor may first determine a lead-group assignment for a given lead and may then apply the corresponding score-generation equation, coefficient set, or model parameters to the morphology metrics computed for that lead. In some embodiments, the equations shown in FIG. 2 represent a logistic-regression-based implementation in which magnitude-related information and symmetry-related information are each transformed and then combined to produce a HATW score. In other embodiments, the equations shown in FIG. 2 may be understood as exemplary of a broader class of lead-group-specific score-generation models, and the present disclosure is not limited to only the exact coefficients or mathematical expressions depicted.

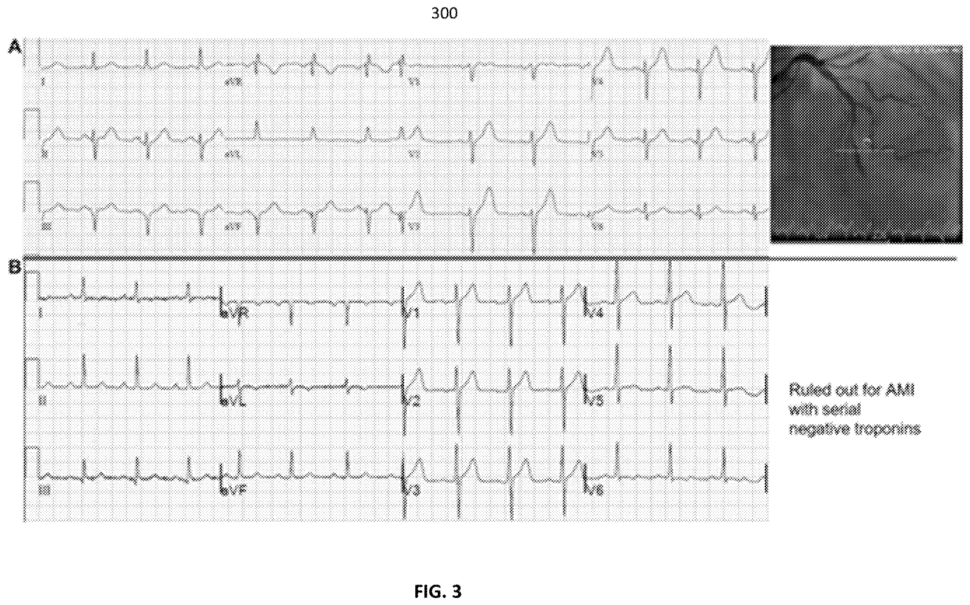

FIG. 3 shows example clinical ECGs that illustrate application of the disclosed HATW framework to different waveform presentations. In the exemplary embodiment, FIG. 3 includes a first ECG example corresponding to a true occlusion myocardial infarction (OMI) case in which hyperacute T waves are identified by the HATW score, and a second ECG example corresponding to a non-OMI case in which some ST elevation may be present but the waveform does not satisfy the disclosed HATW criteria. In various embodiments, the example OMI case exhibits relatively large T-wave area compared to QRS size together with increased symmetry in relevant leads, whereas the comparison case exhibits less of the morphology pattern captured by the disclosed scoring approach. FIG. 3 therefore provides a side-by-side illustration of how the disclosed framework distinguishes between an ECG with clinically significant hyperacute morphology and an ECG that may appear superficially concerning but does not exhibit the same quantified morphology profile.

In some embodiments, FIG. 3 may be understood as illustrating the practical effect of applying the processor-executed morphology analysis described with respect to FIGS. 1 and 2 to full clinical ECG data. The figure is not limited to only the specific cases shown, but rather demonstrates that the disclosed HATW score may identify waveform patterns associated with acute coronary occlusion even where conventional ST-segment elevation criteria are absent or less pronounced, while also avoiding positive classification in at least some non-OMI patterns having more benign or non-hyperacute morphology. The ECGs in FIG. 3 are exemplary and are included to illustrate representative use of the disclosed techniques rather than to limit the disclosure to any particular patient presentation, coronary territory, or waveform appearance.

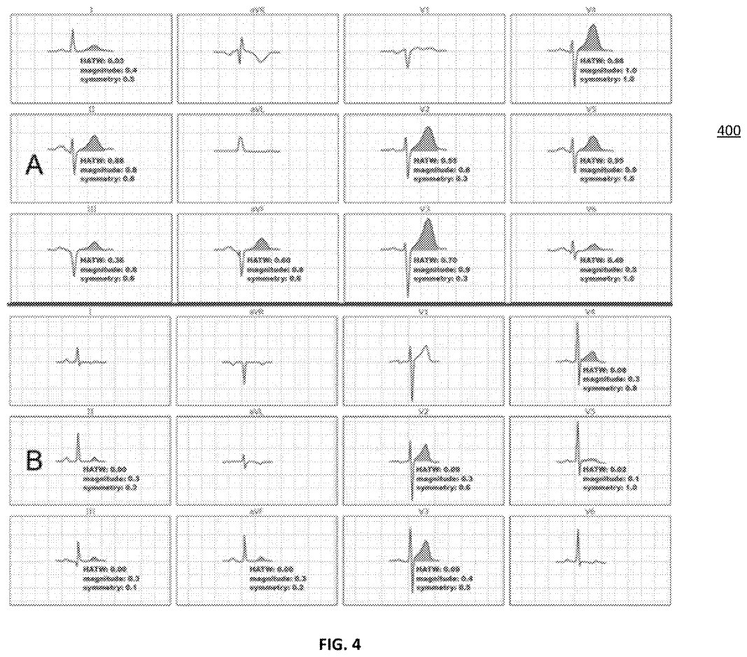

FIG. 4 shows median beat representations corresponding to the ECG examples of FIG. 3 and illustrates how per-lead scores may be associated with individual lead waveforms. In the exemplary embodiment, FIG. 4 displays, for each relevant lead, a HATW score together with a magnitude score and a symmetry score, and visually differentiates leads that satisfy or fail to satisfy a selected score threshold. As explained here, this type of representation may be generated after constructing median beat waveforms from one or more cardiac cycles, thereby reducing noise and providing a stable basis for delineating waveform boundaries and computing morphology metrics. FIG. 4 therefore serves both as an example of preprocessing output and as an example of how lead-level component scores may be presented in connection with the underlying ECG waveform.

In some embodiments, FIG. 4 may be understood as supporting implementations in which a processor computes lead-level component metrics, generates a lead-level HATW score, and then outputs those values in a user-facing or machine-readable format. Visual differentiation in the figure may indicate whether a lead-level score exceeds a threshold, contributes to an ECG-level decision, or otherwise warrants emphasis. The specific coloring, thresholds, layout, and score presentation style shown in FIG. 4 are exemplary only. More generally, FIG. 4 illustrates that the disclosed techniques may present not only a final per-lead or per-ECG result, but also intermediate morphology information that contributes to transparency, interpretability, review, and downstream workflow integration.

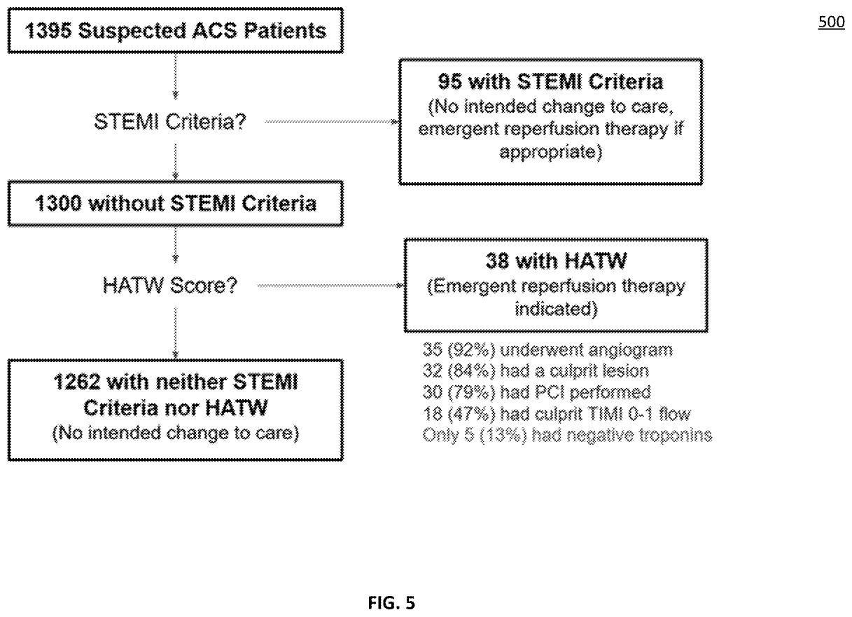

FIG. 5 depicts an example outcome flow associated with application of the disclosed HATW score in a clinical decision pathway. In the illustrated embodiment, the flow organizes ECG cases or patient cases according to whether STEMI criteria are present, whether the HATW score satisfies one or more positivity conditions, and the corresponding downstream clinical outcome categories. Thus, FIG. 5 provides an example of how the disclosed score may be used not merely as an isolated waveform-analysis output, but as a processor-generated indicator that can be incorporated into a reperfusion-oriented evaluation workflow. In some embodiments, the flow shown in FIG. 5 may be generated from a validation data set, registry data set, retrospective cohort, prospective cohort, or other collection of ECG cases and associated clinical outcomes.

In some embodiments, FIG. 5 illustrates that the disclosed HATW framework may identify a subset of clinically significant cases outside of those captured by conventional STEMI criteria alone. For example, the flow may show cases that are STEMI-negative but HATW-positive and may further indicate how such cases distribute across outcome categories such as confirmed culprit-lesion myocardial infarction, alternative acute myocardial infarction definitions, rule-out populations, or other adjudicated clinical groups. In this way, FIG. 5 supports embodiments in which the HATW score functions as a STEMI-equivalent indicator, a triage input, an alerting trigger, or an additional decision-support signal used to prioritize expedited review or intervention. The specific counts, branches, and cohort composition shown in FIG. 5 are exemplary only and may vary depending on the study population, outcome definition, threshold selection, and deployment setting.

In some embodiments, the flow represented in FIG. 5 may also be used to calibrate or select operating thresholds for deployment. For example, one or more threshold conditions may be selected to balance specificity and sensitivity in a target population, and the resulting pathway distribution may be reviewed to determine whether the score is suitable for use as a high-specificity alerting criterion, a screening criterion, or a supplemental indicator used in conjunction with STEMI criteria or other ECG analysis outputs. Accordingly, FIG. 5 is useful not only as an example validation-oriented figure, but also as a representation of how score outputs produced by the disclosed techniques may map into downstream clinical workflow categories.

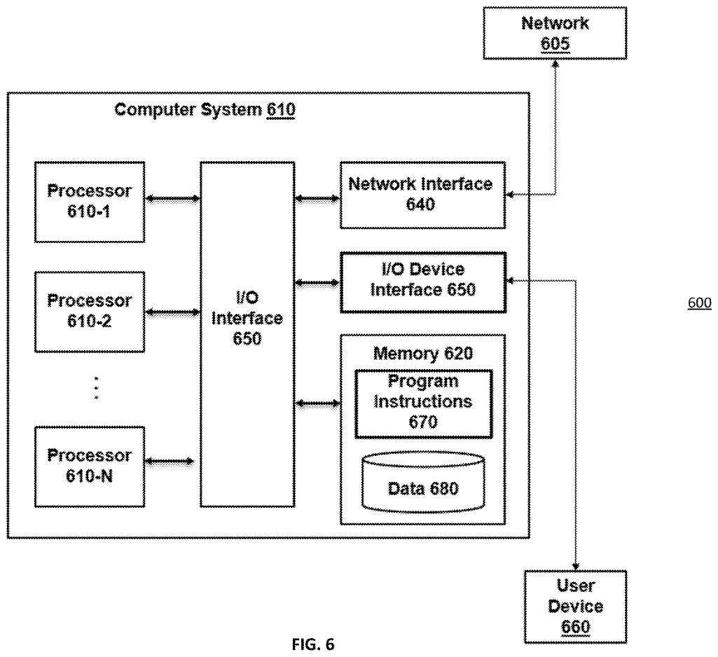

FIG. 6 is a block diagram of an example computing system on which the systems and methods described herein may be executed. In some embodiments, FIG. 6 may correspond to a general-purpose or special-purpose computing environment including one or more processors, memory, input/output interfaces, network interfaces, and associated program instructions or stored data structures. The computing system of FIG. 6 may execute one or more portions of the HATW detection workflow, including ECG data ingestion, waveform segmentation, morphology metric computation, score generation, visualization, alerting, model training, serial comparison, and downstream data exchange. The arrangement shown in FIG. 6 is illustrative and may be implemented as a local device, a remote server, a cloud-based compute instance, a distributed computing environment, or a combination thereof.

In some embodiments, FIG. 6 may also support deployment flexibility across different clinical and technical environments. For example, some processing stages may execute on an ECG acquisition device, while other stages may execute on a hospital server, a remote analysis platform, or another network-accessible system. Likewise, storage associated with FIG. 6 may maintain raw ECG waveforms, segmented waveform objects, metric values, model parameters, thresholds, audit data, historical ECGs, or visualization state information. FIG. 6 is therefore useful as a general computing-environment figure that supports the processor-executed implementation of the various techniques described throughout the present disclosure.

Some embodiments may execute the above operations on a computer system, such as the computer system of FIG. 6, which is a diagram that illustrates a computing system 600 in accordance with embodiments of the present techniques. Various portions of systems and methods described herein, may include or be executed on one or more computer systems similar to computing system 600. Further, processes and modules described herein may be executed by one or more processing systems similar to that of computing system 600.

Computing system 600 may include one or more processors (e.g., processors 610a-610n) coupled to system memory 620, an input/output I/O device interface 630, and a network interface 640 via an input/output (I/O) interface 650. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 600. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 620). Computing system 600 may be a uni-processor system including one processor (e.g., processor 610a), or a multi-processor system including any number of suitable processors (e.g., 610a-610n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus may also be implemented as, special purpose logic circuitry, e.g., an FPGA (field programmable gate array) or an ASIC (application specific integrated circuit). Computing system 600 may include a plurality of computing devices (e.g., distributed computer systems) to implement various processing functions.

I/O device interface 630 may provide an interface for connection of one or more I/O devices 660 to computer system 600. I/O devices may include devices that receive input (e.g., from a user) or output information (e.g., to a user). I/O devices 660 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 660 may be connected to computer system 600 through a wired or wireless connection. I/O devices 660 may be connected to computer system 600 from a remote location. I/O devices 660 located on remote computer system, for example, may be connected to computer system 600 via a network and network interface 640.

Network interface 640 may include a network adapter that provides for connection of computer system 600 to a network. Network interface 640 may facilitate data exchange between computer system 600 and other devices connected to the network. Network interface 640 may support wired or wireless communication. The network may include an electronic communication network, such as the Internet, a local area network (LAN), a wide area network (WAN), a cellular communications network, or the like.

System memory 620 may be configured to store program instructions 670 or data 680. Program instructions 670 may be executable by a processor (e.g., one or more of processors 610a-610n) to implement one or more embodiments of the present techniques. Instructions 670 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 620 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 620 may include a non-transitory computer readable storage medium that may have program instructions stored thereon that are executable by a computer processor (e.g., one or more of processors 610a-610n) to cause the subject matter and the functional operations described herein. A memory (e.g., system memory 620) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 650 may be configured to coordinate I/O traffic between processors 610a-610n, system memory 620, network interface 640, I/O devices 660, and/or other peripheral devices. I/O interface 650 may perform protocol, timing, or other data transformations to convert data signals from one component (e.g., system memory 620) into a format suitable for use by another component (e.g., processors 610a-610n). I/O interface 650 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 600 or multiple computer systems 600 configured to host different portions or instances of embodiments. Multiple computer systems 600 may provide for parallel or sequential processing/execution of one or more portions of the techniques described herein.

Those skilled in the art will appreciate that computer system 600 is merely illustrative and is not intended to limit the scope of the techniques described herein. Computer system 600 may include any combination of devices or software that may perform or otherwise provide for the performance of the techniques described herein. For example, computer system 600 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 600 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 600 may be transmitted to computer system 600 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

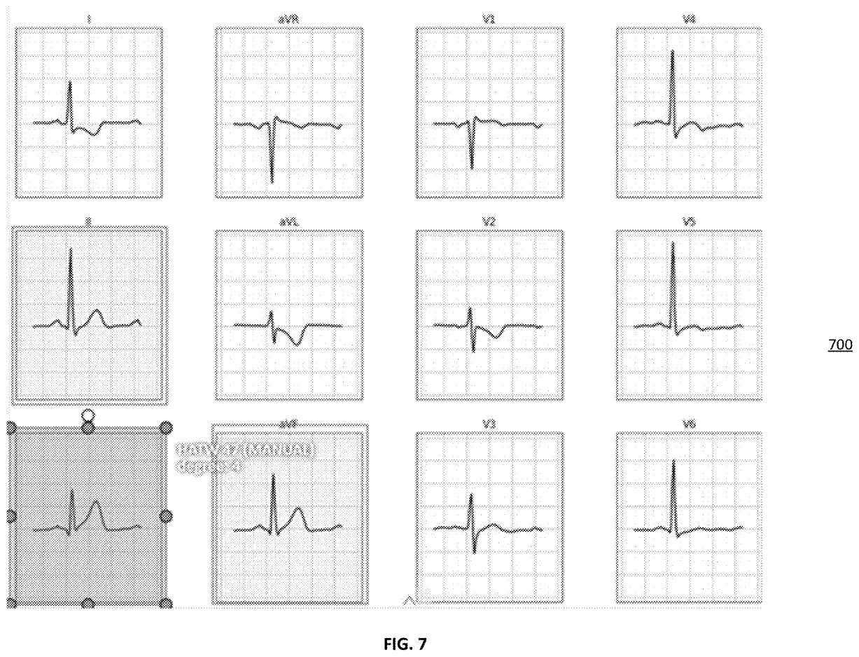

FIG. 7 shows an example ECG with expert annotations marking hyperacute T waves in specific leads. In the exemplary embodiment, FIG. 7 illustrates how one or more expert reviewers may identify lead-level presence or absence of hyperacute morphology for use in derivation, validation, adjudication, or training workflows. In some embodiments, such annotations may be applied on a per-lead basis and may serve as supervisory input for training a model to recognize hyperacute morphology from processor-computed waveform metrics. FIG. 7 therefore provides a representative example of the annotation process underlying at least some embodiments of the training framework described herein.

In some embodiments, the annotations shown in FIG. 7 may include binary markings, severity gradations, region markers, lead labels, or other indicators associated with hyperacute morphology. The figure is illustrative only and does not require that every embodiment use manual expert annotation, identical annotation conventions, or the same annotation resolution. More generally, FIG. 7 demonstrates that the disclosed techniques may be grounded in annotated ECG data and that expert-identified hyperacute morphology may be represented in a structured form suitable for model training, threshold tuning, interrater analysis, or other processor-executed workflows.

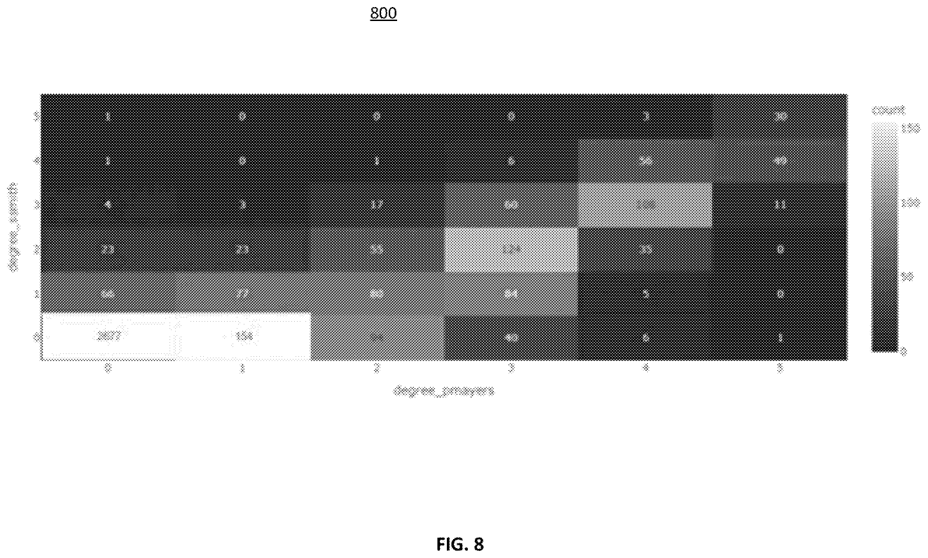

FIG. 8 is a heatmap illustrating interrater reliability between ECG experts for hyperacute T-wave annotation. In the exemplary embodiment, FIG. 8 visually represents the degree of agreement and disagreement between expert reviewers across one or more annotation categories or severity levels. In some embodiments, this type of figure helps demonstrate that expert evaluation of hyperacute morphology may serve as a reproducible annotation signal, even though certain disagreements may cluster around borderline cases. FIG. 8 is therefore useful in supporting embodiments in which expert annotations are used as training labels, calibration references, or validation signals for one or more HATW models.

FIG. 8 is included as an evidentiary and explanatory figure rather than as a required runtime component of the disclosed system. In some embodiments, interrater-agreement information may be used to select training examples, assign weights, identify ambiguous cases, or characterize label reliability. In other embodiments, no explicit interrater heatmap need be generated during runtime. More generally, FIG. 8 illustrates that the disclosed training and annotation framework may incorporate structured expert agreement information and that the derived scoring techniques may be based on annotations having measurable reproducibility.

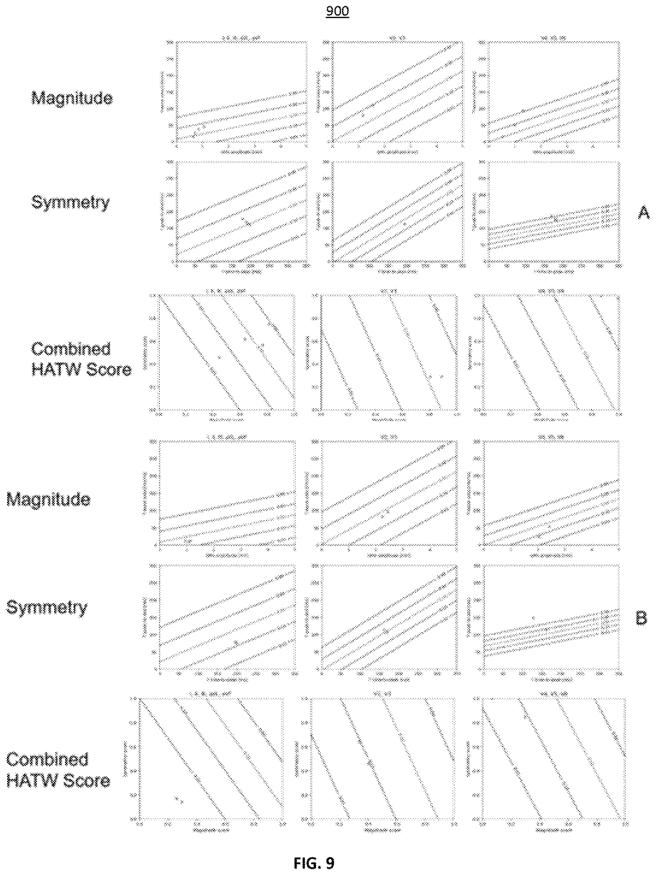

FIG. 9 includes scatter plots showing per-lead scores for one or more example cases superimposed on lead-group-specific decision boundaries. In the exemplary embodiment, FIG. 9 provides a visualization of how individual lead-level measurements or scores for selected ECG examples are positioned relative to the separation regions or threshold boundaries associated with different lead-group-specific models. Thus, FIG. 9 may be understood as a more case-specific companion to FIG. 2, illustrating how actual example leads map into the score space and how those leads are classified relative to the learned or defined decision structure for a given lead group.

In some embodiments, FIG. 9 supports implementations in which a processor not only computes lead-level scores but also evaluates those scores relative to lead-group-specific model behavior, decision thresholds, or boundary surfaces. The figure may also support visualization embodiments in which component scores, confidence indicators, or classification context are displayed for review. The particular example cases, boundary presentations, and plotted score values shown in FIG. 9 are exemplary only. More broadly, FIG. 9 illustrates that the disclosed HATW framework may provide interpretable lead-level positioning within a morphology-based decision space rather than merely emitting an opaque binary result.

FIG. 10 depicts an illustrative ECG system 1000 for identifying hyperacute T-waves and for implementing one or more of the systems and methods described herein. In some embodiments, system 1000 may be deployed in a clinical environment, a remote monitoring environment, a portable or wearable ECG ecosystem, a cloud-based analysis environment, or any combination thereof. System 1000 may include hardware, software, communications infrastructure, and stored data structures that cooperate to receive ECG waveform data, process the waveform data to identify relevant signal components, compute one or more T-wave morphology metrics, generate one or more HATW-related scores or classifications, and provide outputs suitable for visualization, alerting, downstream model integration, serial comparison, and clinical workflow support. As shown, system 1000 may include network 1005, ECG device(s) 1020, user device(s) 1030, clinical system(s) 1040, database(s) 1050, and server 1010, which may host HATW detection application 1011. In some embodiments, each of the components of system 1000 may communicate over network 1005.

The arrangement shown in FIG. 10 is illustrative rather than limiting. In some embodiments, functions shown as occurring within server 1010 may be distributed across multiple servers, local ECG devices, edge-processing devices, clinical workstations, or other computing platforms. In some embodiments, one or more modules shown within HATW detection application 1011 may be combined, omitted, replicated, or reorganized. Likewise, one or more external systems shown in FIG. 10 may be integrated into a single device or may be implemented as separate devices or services communicating through one or more wired, wireless, or networked interfaces. The particular organization shown in FIG. 10 is therefore intended to illustrate one example of a processor-executed architecture that may be used to implement the disclosed techniques.

Network 1005 may represent one or more communication infrastructures that facilitate exchange of ECG waveform data, derived morphology data, threshold information, model parameters, visualization data, alerts, and other information among components of system 1000. In various embodiments, network 1005 may include one or more public networks, private networks, hospital intranets, cloud networks, local area networks, wide area networks, cellular networks, wireless telemetry links, or combinations thereof. For example, network 1005 may include a hospital network connecting one or more ECG machines, clinical workstations, and server resources, while also supporting communication with remote services, mobile devices, or cloud-hosted analysis platforms. In some embodiments, network 1005 may support both wired and wireless communication paths depending on the source of the ECG data and the location of the processing resources.

In some embodiments, network 1005 may carry ECG signal data from ECG device(s) 1020 to server 1010 for processing by HATW detection application 1011. In some embodiments, network 1005 may also carry processed outputs from server 1010 to user device(s) 1030, clinical system(s) 1040, or database(s) 1050. Such outputs may include raw or preprocessed waveform data, delineation results, lead-level morphology metrics, HATW scores, ECG-level determinations, culprit-localization outputs, temporal comparison outputs, alert messages, or downstream pipeline data. In some embodiments, network 1005 may further support bi-directional communication, such as transmission of historical ECG data from database(s) 1050 to server 1010 for serial comparison, delivery of user acknowledgments or review inputs from user device(s) 1030, or receipt of configuration updates, model versions, or workflow data from one or more connected systems. Thus, network 1005 may provide the communication layer through which the various processing, storage, visualization, and integration operations of system 1000 are coordinated.

In some embodiments, server 1010 may execute a software system referred to herein as HATW detection application 1011. HATW detection application 1011 may include a coordinated set of processor-executed modules configured to receive ECG signal data, prepare and segment waveform data, determine whether one or more leads are eligible for HATW analysis, compute one or more morphology metrics, generate one or more HATW-related scores or classifications, evaluate one or more threshold conditions, and provide one or more outputs for visualization, alerting, serial comparison, culprit localization, or downstream model integration. In some embodiments, application 1011 may operate in real time or near real time as ECG data is acquired. In some embodiments, application 1011 may operate on previously stored ECG recordings, batched ECG studies, median beat representations, or other waveform-derived data structures. In some embodiments, application 1011 may be deployed on a single physical or virtual server. In other embodiments, portions of application 1011 may be distributed across multiple machines, services, or execution environments that cooperate to carry out the disclosed operations.

In some embodiments, server 1010 may receive ECG waveform data from one or more external sources, including ECG device(s) 1020, database(s) 1050, user device(s) 1030, or clinical system(s) 1040, and may maintain in memory or storage one or more associated data structures used during analysis. Such data structures may include raw waveform samples, filtered waveform data, segmented waveform intervals, median beat representations, lead identifiers, lead-group assignments, eligibility flags, morphology metric values, intermediate score values, model parameters, threshold settings, historical ECG references, audit data, and output records. During execution, server 1010 may schedule or invoke the modules of HATW detection application 1011 sequentially, concurrently, or conditionally depending on data availability, configuration settings, deployment mode, or workflow context. In some embodiments, one or more modules may exchange intermediate outputs through shared memory, serialized objects, message queues, API calls, or database-backed state records.

The module arrangement shown within HATW detection application 1011 is illustrative and not limiting. In some embodiments, additional modules may be included. In some embodiments, fewer modules may be used. In some embodiments, one or more modules shown separately in FIG. 10 may be combined into a single processing component, subdivided into multiple cooperating submodules, omitted for a particular deployment, or replaced by functionally similar logic organized in a different manner. For example, one embodiment may combine morphology feature extraction and score generation into a single inference module, while another embodiment may separate segmentation, eligibility filtering, and metric computation into distinct microservices or processing stages. Similarly, one or more optional modules, such as serial comparison, culprit localization, or downstream pipeline integration, may be omitted in deployments that do not use those functions. Accordingly, the module organization shown in FIG. 10 should be understood as one example of an architecture capable of implementing the disclosed techniques, rather than as a required or exhaustive enumeration of all possible system configurations.

HATW detection application 1011 may be a processor-executed software application that coordinates the operations used to identify hyperacute T-wave morphology from ECG data and to generate one or more associated outputs. In some embodiments, application 1011 may receive ECG signal data from one or more acquisition devices, stored ECG repositories, clinical systems, or user-facing systems, and may transform that data through a sequence of processing stages into one or more lead-level or ECG-level results. Such results may include delineated waveform components, morphology metric values, intermediate score values, HATW scores, threshold determinations, serial comparison outputs, culprit-localization outputs, visualization data, alert data, or downstream integration outputs. In some embodiments, application 1011 may operate continuously as a service that processes incoming ECG data streams. In some embodiments, application 1011 may instead be invoked on demand in response to receipt of a stored ECG study, a user request, a clinical workflow trigger, or a batch-processing job.

In some embodiments, application 1011 may maintain execution state for a given ECG record or analysis session across multiple processing stages. For example, application 1011 may associate a received ECG with one or more internal identifiers, lead labels, waveform segmentation results, eligibility determinations, feature vectors, model parameters, threshold settings, and output records so that intermediate and final results remain linked throughout the analysis workflow. In some embodiments, application 1011 may also access or maintain configuration profiles that govern model selection, lead-group definitions, threshold logic, alert criteria, visualization preferences, device-specific calibration parameters, or deployment-specific workflow behavior. Such information may be maintained in local memory, database-backed storage, configuration files, remote services, or combinations thereof. Through this coordinated execution, application 1011 may function as the central control layer through which the system receives ECG data, executes HATW analysis, manages intermediate state, and delivers outputs to other modules, devices, or external systems.

In some embodiments, ECG ingestion module 1012 may be configured to receive ECG signal data from one or more upstream sources and to convert the received data into an internal representation suitable for subsequent processing by HATW detection application 1011. The received ECG signal data may include waveform samples from one or more ECG leads together with associated metadata such as lead identifiers, sampling rate information, acquisition timestamps, patient or study identifiers, device identifiers, calibration data, signal quality indicators, rhythm labels, or workflow context information. In some embodiments, module 1012 may receive the ECG signal data directly from ECG device(s) 1020 at or near the time of acquisition. In some embodiments, module 1012 may receive previously stored ECG recordings from database(s) 1050, user-initiated uploads or requests from user device(s) 1030, or study exports, alert-triggering records, or other ECG-related inputs from clinical system(s) 1040. Thus, module 1012 may serve as the entry point through which waveform data enters the HATW analysis pipeline.

In some embodiments, module 1012 may support multiple ingestion modes. For example, module 1012 may receive ECG data through a direct device interface, a network-based application programming interface, a file transfer mechanism, a message queue, a streaming telemetry connection, a database query, or another machine-to-machine communication pathway. In some embodiments, module 1012 may accept data encoded in one or more ECG formats, including waveform arrays, median beat files, XML-encoded ECG studies, proprietary device export formats, structured database records, or other digital representations of cardiac electrical activity. When incoming data is received, module 1012 may parse the data, identify the available leads, determine whether the ECG is a full 12-lead study or a reduced-lead recording, and instantiate one or more internal data objects representing the ECG study, the individual lead waveforms, and associated metadata. In some embodiments, those data objects may be stored in volatile memory for immediate downstream processing. In some embodiments, module 1012 may persist the data objects to local or remote storage to support deferred processing, re-analysis, serial comparison, audit logging, or workflow continuity.

Module 1012 may additionally perform one or more intake-validation operations before passing data downstream. In some embodiments, module 1012 may verify that the received ECG data includes sufficient waveform content, recognizable lead labeling, and usable signal formatting for further analysis. In some embodiments, module 1012 may determine whether required metadata fields are present, whether the waveform length and sampling resolution satisfy one or more analysis criteria, whether duplicate or corrupted records are present, and whether the data should be associated with an existing study, patient, or serial-comparison record. When data irregularities are detected, module 1012 may reject the record, quarantine the record, request retransmission, flag the record for degraded-confidence processing, or annotate the record so that later modules may take the irregularities into account. In some embodiments, module 1012 may also assign one or more internal identifiers to the ECG study and to the associated lead-level waveform objects so that later segmentation, scoring, alerting, and output operations may be linked back to the originating ECG instance.

In some embodiments, module 1012 may enrich the incoming ECG data with additional context before transferring control to signal processing and waveform segmentation module 1013. For example, module 1012 may append deployment-specific configuration information, identify an acquisition-device type, determine whether device-specific recalibration parameters should be used, associate the ECG with a prior ECG for the same subject, or tag the ECG as belonging to a particular workflow context such as emergency department triage, serial monitoring, outpatient screening, or wearable-device review. In some embodiments, module 1012 may additionally record receipt times, source-system identifiers, transmission-channel details, or data-integrity markers for audit or troubleshooting purposes. Once the ECG data has been received, parsed, validated, and represented in an internal format, module 1012 may provide the resulting waveform and metadata objects to signal processing and waveform segmentation module 1013 for additional preparation and component delineation.

In some embodiments, signal processing and waveform segmentation module 1013 may be configured to transform ingested ECG waveform data into a form suitable for morphology analysis by reducing signal artifacts, identifying or constructing analysis-ready waveform representations, and delineating one or more waveform intervals relevant to subsequent metric computation. Module 1013 may receive, from ECG ingestion module 1012, one or more waveform objects together with associated metadata such as lead labels, sample timing information, acquisition parameters, and any intake-validation annotations. Based on that input, module 1013 may apply one or more preprocessing operations to improve the stability and reproducibility of later measurements. Such operations may include, for example, filtering, baseline correction, denoising, beat alignment, beat selection, median beat construction, rhythm-consistency assessment, motion-artifact suppression, electrode-contact artifact detection, and other signal-conditioning steps. In some embodiments, module 1013 may operate on raw waveform samples for each lead. In some embodiments, module 1013 may generate one or more derived waveform objects that are then used as the primary input for segmentation and later feature extraction.

In some embodiments, module 1013 may construct a median beat representation for one or more leads. For example, module 1013 may identify multiple cardiac cycles within an ECG recording, align corresponding beats according to one or more fiducial points, and generate a representative beat waveform by combining the aligned beats through a median, trimmed mean, or other aggregation operation. A median beat representation may attenuate transient noise, reduce beat-to-beat variability unrelated to the morphology of interest, and provide a more stable signal for later delineation of QRS and T-wave boundaries. In some embodiments, module 1013 may construct a median beat for each lead independently. In some embodiments, module 1013 may construct a multi-lead median beat set that preserves temporal alignment across leads. In some embodiments, if a sufficient number of stable beats is not available, module 1013 may instead operate on a selected representative beat, on a short segment of beats, or on another waveform representation while optionally recording a reduced-confidence indicator for downstream use.

Module 1013 may also perform waveform segmentation operations that identify one or more ECG components and boundaries used in subsequent morphology calculations. In some embodiments, module 1013 may identify QRS onset, QRS peak, QRS offset, J-point location, T-wave onset region, T-wave peak, and T-wave end for each eligible or potentially eligible lead. Such delineation may be performed using threshold-based logic, derivative-based operators, template matching, adaptive windowing, machine learning-assisted segmentation, or other segmentation routines suitable for ECG waveform analysis. In some embodiments, module 1013 may maintain one or more per-lead segmentation data structures storing the sample indices, time coordinates, amplitudes, confidence values, or quality flags associated with the identified waveform boundaries. Those segmentation structures may then be passed to lead eligibility filter 1014 and morphology feature extraction module 1015 so that the later modules may compute metrics from explicit, processor-identified waveform intervals rather than from undifferentiated raw signal data. In some embodiments, if segmentation confidence is low for a particular lead or interval, module 1013 may flag that lead for exclusion, alternate processing, or reduced-confidence scoring downstream.

In some embodiments, module 1013 may also generate one or more auxiliary outputs that support later system behavior. For example, module 1013 may compute signal quality measures, identify whether a lead exhibits excessive noise or baseline drift, detect whether one or more beats should be excluded from median beat construction, or determine whether the overall waveform morphology is likely to be distorted by conduction abnormalities or acquisition artifacts. Such outputs may be stored as lead-level or study-level metadata and may influence later eligibility filtering, threshold evaluation, visualization, or alerting behavior. Once the waveform data has been conditioned and the relevant ECG intervals have been segmented, module 1013 may provide the resulting processed waveform objects, segmentation boundaries, and quality annotations to lead eligibility filter 1014 for determination of whether one or more leads are suitable for HATW scoring.

In some embodiments, lead eligibility filter 1014 may be configured to determine whether one or more ECG leads are suitable for HATW analysis before morphology metrics are computed and before one or more scores are generated. Module 1014 may receive, from signal processing and waveform segmentation module 1013, one or more processed waveform representations together with segmentation boundaries, timing data, signal-quality indicators, and other lead-level metadata. Using that information, module 1014 may evaluate whether the waveform content for a given lead is sufficiently well-defined and sufficiently compatible with the intended scoring framework to support reliable morphology measurement. In some embodiments, this filtering stage may reduce instability in later score generation by excluding leads having waveform characteristics that would render magnitude, symmetry, or related morphology metrics unreliable or uninformative. In some embodiments, module 1014 may produce a per-lead eligibility flag, one or more reason codes, and optionally one or more degraded-confidence indicators for downstream modules.

In an exemplary embodiment, module 1014 may evaluate one or more lead eligibility criteria associated with waveform morphology and signal definition. Such criteria may include, for example, whether the lead corresponds to a lead type intended for analysis, whether the QRS duration is below a selected threshold, whether the total QRS amplitude satisfies a minimum amplitude condition, whether the T-wave amplitude is sufficiently positive and sufficiently well-defined, whether the onset-to-peak and peak-to-end portions of the T wave satisfy minimum duration criteria, and whether the T-wave shape is non-inverted and non-biphasic according to one or more morphological rules. In some embodiments, the module may determine these criteria using segmentation points identified by module 1013, such as QRS start and end positions, J-point location, T-wave peak location, and T-wave end location, together with amplitude and duration values computed from the waveform samples. In some embodiments, module 1014 may also consider signal quality indicators, rhythm irregularity indicators, or device-specific constraints when determining whether a lead should proceed to feature extraction.

The specific eligibility rules applied by module 1014 may vary by embodiment and deployment context. In some embodiments, module 1014 may apply the exemplary lead-level criteria associated with the disclosed HATW framework, including narrow-QRS requirements, minimum QRS amplitude requirements, minimum T-wave amplitude requirements, minimum T-wave interval requirements, and one or more rules excluding leads having inverted or biphasic T-wave morphology. In some embodiments, one or more leads, such as aVR or another lead not used in a particular scoring configuration, may be excluded categorically. In other embodiments, module 1014 may use modified thresholds, omit one or more criteria, or relax one or more exclusion rules for particular device types, reduced-lead systems, wearable environments, serial-monitoring contexts, or alternative model configurations. In some embodiments, module 1014 may apply lead-group-specific eligibility logic, and in some embodiments eligibility filtering may be reduced, replaced, or omitted entirely in favor of a model configured to operate on a broader or less constrained input set. Accordingly, the eligibility filtering performed by module 1014 should be understood as illustrative of one robust implementation for improving score reliability, rather than as a mandatory prerequisite for every embodiment.

Once the eligibility determination has been made, module 1014 may route leads differently depending on the outcome. In some embodiments, leads marked as eligible may be passed to morphology feature extraction module 1015 together with the associated waveform segments and segmentation metadata. In some embodiments, leads marked as ineligible may be excluded from HATW scoring, flagged for alternate processing, retained for recordkeeping, or displayed with an indication that score generation was not performed for that lead. Module 1014 may additionally store or transmit one or more eligibility-related outputs, such as a count of eligible leads, a lead-group eligibility summary, one or more exclusion reasons, or an ECG-level flag indicating whether sufficient eligible information is available for a multi-lead determination. These outputs may influence threshold and multi-lead evaluation performed later in the pipeline and may also support transparency in visualization, serial comparison, and audit functions.

In some embodiments, morphology feature extraction module 1015 may be configured to compute one or more quantitative waveform descriptors from the eligible ECG leads identified by lead eligibility filter 1014. Module 1015 may receive, for each lead selected for further analysis, one or more processed waveform representations, segmentation boundaries, eligibility indicators, and related metadata. Using that information, module 1015 may compute one or more values characterizing T-wave morphology relative to the surrounding ECG waveform. In some embodiments, the resulting values may be stored as lead-level features in one or more data structures associated with the ECG record, and may be provided to HATW scoring module 1016 for score generation. In some embodiments, module 1015 may compute a fixed feature set for each eligible lead. In some embodiments, the computed feature set may vary depending on deployment configuration, lead type, model selection, device type, or workflow context.

In an exemplary embodiment, module 1015 may compute at least a T-wave magnitude metric and a T-wave symmetry metric. The T-wave magnitude metric may quantify a relative size, bulk, or extent of a T-wave with respect to the corresponding QRS complex. For example, module 1015 may determine the area under the T-wave waveform between a J-point and a T-wave end location and may normalize that area using a QRS amplitude value determined from a maximum-to-minimum amplitude range of the QRS complex. The T-wave symmetry metric may quantify the temporal distribution of the T-wave peak within the T-wave interval. For example, module 1015 may determine a first interval extending from the J-point or T-wave onset region to the T-wave peak, determine a second interval extending from the T-wave peak to the T-wave end, and compute a ratio, transformation, or other function of those intervals. In some embodiments, these metrics may be computed directly from sample indices or time values derived from the segmentation output of module 1013. In some embodiments, one or more normalization, clipping, smoothing, or transformation operations may be applied before the feature values are stored or passed downstream.

Module 1015 may additionally compute one or more optional morphology features. In some embodiments, module 1015 may compute an ST-segment depression metric measured relative to a selected baseline reference. In some embodiments, module 1015 may compute a convexity metric characterizing whether the T-wave contour more closely resembles a convex, dome-shaped waveform or another morphology. In some embodiments, module 1015 may compute one or more additional lead-specific or multi-lead features, such as T-wave sub-interval areas, slope ratios, reciprocal-lead relationships, spatial gradients, concordance or discordance indicators, inter-lead feature ratios, or transformed waveform representations derived using signal-processing or dimensionality-reduction techniques. In some embodiments, module 1015 may also receive or generate features derived from fewer than twelve leads, including reduced-lead or wearable-device inputs, and may apply feature-calculation logic calibrated for those configurations. Thus, module 1015 may serve as the stage at which processed ECG waveform content is transformed into one or more structured feature values suitable for model-based scoring, rule-based evaluation, visualization, or downstream model integration.

In some embodiments, module 1015 may store not only the final feature values but also one or more intermediate measurement values used to derive them. For example, module 1015 may maintain area values, amplitude values, interval durations, curvature values, reciprocal-lead references, quality indicators, or confidence scores associated with the extracted features. Such intermediate data may later be used for visualization, auditability, score explanation, model debugging, serial comparison, or threshold tuning. Once the selected morphology features have been computed, module 1015 may provide the resulting feature vectors or other structured feature records to HATW scoring module 1016 for score generation.

In some embodiments, HATW scoring module 1016 may be configured to generate one or more lead-level or ECG-level scores indicative of hyperacute T-wave morphology using the feature values produced by morphology feature extraction module 1015. Module 1016 may receive, for one or more leads, a feature set comprising at least a T-wave magnitude metric and a T-wave symmetry metric and, in some embodiments, one or more optional additional features such as ST-segment depression, convexity, inter-lead relationships, or other derived morphology indicators. Based on those inputs, module 1016 may apply one or more score-generation functions, trained models, rule sets, parameterized equations, or other processor-executed logic to produce a HATW score or another composite morphology score for each analyzed lead. In some embodiments, module 1016 may also produce one or more intermediate scores, such as a magnitude-related sub-score or a symmetry-related sub-score, and may store those intermediate values for later visualization, threshold evaluation, or explanation.

In an exemplary embodiment, module 1016 may implement a score-generation architecture in which magnitude-related information and symmetry-related information are transformed through one or more logistic functions and then combined into a lead-level HATW score. In some embodiments, module 1016 may use separate parameter sets for different lead groups, such as limb leads, right precordial leads, and left precordial leads. Accordingly, module 1016 may first determine a lead-group assignment for a given lead and may then apply a corresponding coefficient set, model instance, or parameterized equation to the features extracted for that lead. In some embodiments, the resulting HATW score may be a continuous numerical value, such as a value bounded between zero and one. In some embodiments, the HATW score may instead be represented on another numerical scale, transformed into a categorical or binary output, or supplemented with one or more confidence or quality indicators.

Module 1016 is not limited to any single model family. In some embodiments, module 1016 may apply logistic regression, a feedforward neural network, a tree-based model, a support vector machine, a random forest, a deep neural network, or another trained or rule-based scoring approach. In some embodiments, module 1016 may generate a score using explicitly computed features such as magnitude and symmetry. In some embodiments, module 1016 may instead operate on a broader set of morphology descriptors or on learned representations derived from waveform data while still producing a score indicative of hyperacute morphology. In some embodiments, module 1016 may also support reduced-lead or device-specific scoring configurations, including models calibrated for wearable or portable ECG systems. In all such cases, module 1016 may generate one or more structured output records containing lead identifiers, lead-group identifiers, score values, optional component scores, optional model identifiers, and associated metadata for use by later modules. Once the lead-level scoring process is complete, module 1016 may provide the score records to threshold and multi-lead evaluation module 1017.

In some embodiments, threshold and multi-lead evaluation module 1017 may be configured to interpret one or more lead-level scores produced by HATW scoring module 1016 and to determine whether one or more ECG-level positivity conditions are satisfied. Module 1017 may receive lead-level HATW scores, optional component scores, lead identifiers, lead-group identifiers, eligibility flags, quality indicators, and related metadata. Using that information, module 1017 may evaluate whether one or more threshold rules, aggregation rules, or multi-lead logic conditions are satisfied by the analyzed ECG. In some embodiments, module 1017 may generate a per-ECG positive or negative determination. In some embodiments, module 1017 may generate a threshold-satisfaction indicator, a count of positive leads, a contiguous-lead grouping result, a spatial distribution result, or other decision-oriented outputs used by downstream modules.

In an exemplary embodiment, module 1017 may determine whether at least two contiguous leads satisfy a selected score condition, such as whether the mean of two contiguous lead scores meets or exceeds a deployment threshold. In some embodiments, the threshold may be selected to favor high specificity, screening sensitivity, or another operating objective. In some embodiments, module 1017 may support configurable threshold values, such as different values for different patient populations, devices, or workflow settings. In some embodiments, module 1017 may use other aggregation logic, including requiring one, three, or more leads, using non-contiguous lead combinations, evaluating reciprocal-lead relationships, applying spatial-gradient constraints, or combining lead-level results with lead-group-specific or ECG-level features. In some embodiments, module 1017 may also generate a confidence or severity indicator based on the number of positive leads, the distribution of positive leads, the distance of one or more scores from a threshold, or the presence of reciprocal or corroborating morphology patterns.

Module 1017 may further support embodiments in which ECG-level evaluation depends on serial data, device configuration, or workflow context. For example, in a reduced-lead environment, module 1017 may apply threshold logic calibrated for fewer available leads. In some embodiments, module 1017 may retain one or more sub-threshold lead-level scores for later serial comparison even if the ECG does not currently satisfy an ECG-level positivity condition. In some embodiments, module 1017 may output one or more decision records indicating why an ECG was classified as positive, negative, indeterminate, or insufficient for determination. Such outputs may include the positive leads, the threshold used, the aggregation mode applied, and any associated rationale or confidence markers. Once module 1017 has evaluated the lead-level results, it may provide the determination and supporting data to output, alert, and visualization module 1018 and, where appropriate, to serial comparison and culprit localization module 1019.

In some embodiments, output, alert, and visualization module 1018 may be configured to generate one or more user-facing, machine-readable, or system-facing outputs based on the lead-level and ECG-level results generated upstream in the HATW analysis pipeline. Module 1018 may receive one or more lead-level scores, ECG-level determinations, threshold-satisfaction results, component metrics, quality indicators, serial-comparison data, workflow flags, and related metadata. Based on those inputs, module 1018 may generate output objects suitable for display, alerting, transmission, logging, or integration with one or more external systems. In some embodiments, the outputs generated by module 1018 may include a per-lead HATW score, a per-ECG positive or negative determination, a confidence indicator, an OMI-likelihood indicator, a STEMI-equivalent indication, a clinical recommendation, or a structured data payload for downstream systems.

In some embodiments, module 1018 may generate visualization outputs associated with one or more ECG waveforms. For example, module 1018 may produce a display representation in which HATW scores are overlaid on or displayed adjacent to corresponding lead waveforms, and may apply color-coding, highlighting, icons, or other visual differentiation to indicate whether a lead score or ECG-level result satisfies one or more thresholds. In some embodiments, module 1018 may additionally present one or more component values, such as per-lead magnitude scores, symmetry scores, ST-depression scores, convexity scores, or confidence indicators. In some embodiments, module 1018 may generate a heatmap, lead-array visualization, trend display, decision-boundary display, or other graphical arrangement supporting review and interpretation of score results. In some embodiments, visualization data generated by module 1018 may be rendered on user device(s) 1030, clinical system(s) 1040, local displays associated with ECG device(s) 1020, or other interfaces.

Module 1018 may also generate one or more alerts or notifications. In some embodiments, when an ECG-level determination or score satisfies one or more configured conditions, module 1018 may generate an alert directed to a clinician, technician, monitoring station, mobile device, or clinical information system. Such an alert may include an audible alert, a visual alert, a push notification, an electronic message, a task-queue entry, or another electronically generated notification. In some embodiments, the alert may include one or more supporting details, such as positive leads, lead-level scores, suspected OMI likelihood, a STEMI-equivalent indication, component score breakdowns, serial trend information, or a recommendation for expedited review, catheterization evaluation, reperfusion assessment, or another clinical action. In some embodiments, module 1018 may further generate structured outputs for storage, audit, or later re-analysis. Thus, module 1018 may function as the principal outward-facing stage through which the analysis results of the disclosed system are rendered actionable, interpretable, and communicable.

In some embodiments, serial comparison and culprit localization module 1019 may be configured to perform higher-level interpretation functions based on multiple ECGs from the same subject and/or on spatial distributions of lead-level scores within a single ECG. Module 1019 may receive one or more current ECG analysis records from upstream modules, together with one or more historical ECG records, lead-level score distributions, threshold outcomes, and associated metadata. Using that information, module 1019 may compare the current ECG analysis results with prior results to identify temporal changes, and may evaluate the spatial arrangement of elevated lead-level scores to infer a likely anatomical pattern or culprit vessel indication. In some embodiments, module 1019 may operate automatically whenever a prior ECG is available. In some embodiments, module 1019 may be invoked only in particular workflow contexts, such as serial chest-pain monitoring, emergency department re-evaluation, or specialist review.

For serial comparison, module 1019 may align a current ECG record with one or more prior ECG records associated with the same subject, encounter, or monitoring session. In some embodiments, the module may compare current and prior lead-level HATW scores, ECG-level determinations, morphology metrics, threshold margins, or other outputs to determine whether a new hyperacute pattern has appeared, whether an existing pattern has increased or decreased, or whether one or more leads have crossed a relevant threshold over time. In some embodiments, module 1019 may generate a temporal trend output, a rise/fall indicator, a change magnitude value, or a serial alert when a clinically significant difference is detected. In some embodiments, module 1019 may incorporate timing information, signal quality data, or lead correspondence logic to ensure that comparisons are made appropriately across serial ECG studies.

For culprit localization, module 1019 may analyze the spatial distribution of elevated scores or features across multiple leads. In some embodiments, module 1019 may determine whether elevated scores cluster in a lead region associated with a particular coronary territory, may evaluate reciprocal or concordant lead patterns, and may generate an output identifying a suspected culprit coronary artery or anatomical region. In some embodiments, the module may use a rule-based approach, a score-aggregation approach, or another model-based localization routine. In some embodiments, the localization output may be appended to one or more alerts, visualization records, or workflow messages generated by module 1018. In some embodiments, module 1019 may omit localization when the lead pattern is ambiguous, when insufficient leads are available, or when the deployment context does not use that functionality. Thus, module 1019 may extend the disclosed HATW framework beyond isolated score generation to include temporal interpretation and anatomical inference based on the pattern of processor-generated results.

In some embodiments, pipeline interface module 1021 may be configured to provide outputs of HATW detection application 1011 to one or more downstream systems, models, services, or workflows. Module 1021 may receive one or more lead-level scores, ECG-level determinations, morphology features, serial-comparison outputs, localization outputs, alerts, visualization-ready data objects, and related metadata from one or more upstream modules. Based on those inputs, module 1021 may generate one or more structured interface payloads that can be consumed by downstream diagnostic models, automated ECG interpretation systems, clinical information systems, electronic health record platforms, analytics environments, or other software components. In some embodiments, module 1021 may serve as the principal integration layer through which HATW-related outputs are exported beyond the immediate HATW analysis pipeline.

In some embodiments, module 1021 may provide HATW scores or related outputs as features to a downstream diagnostic model. For example, module 1021 may transmit one or more lead-level or ECG-level HATW outputs together with other ECG-derived, demographic, laboratory, or clinical context variables to a composite diagnostic model that produces an ischemia, OMI, or broader cardiac-risk output. In some embodiments, module 1021 may also provide thresholded HATW labels or score-derived targets for use in training downstream machine learning systems, including systems that operate directly on raw waveform data during inference without explicitly computing the disclosed morphology metrics. In some embodiments, module 1021 may support batch export, real-time API calls, message-queue publication, file-based export, or another machine-readable transmission mechanism suitable for integration with external processing pipelines.

Module 1021 may also support interoperability with clinical and enterprise systems. In some embodiments, module 1021 may package outputs for delivery to electronic health record systems, diagnostic workstations, rules engines, alert-routing services, remote review portals, or analytics systems. In some embodiments, module 1021 may attach metadata identifying model versions, threshold settings, lead configurations, timestamps, quality flags, or source-study identifiers so that downstream recipients can interpret and manage the outputs appropriately. In some embodiments, module 1021 may also receive acknowledgments, status information, or workflow responses from downstream systems and may provide that information to other components of application 1011 for logging, audit, or workflow-continuation purposes. Thus, module 1021 may allow the disclosed HATW analysis to function not only as a stand-alone scoring engine, but also as a component within broader computational, clinical, and machine-learning pipelines.

In some embodiments, ECG device(s) 1020 may include one or more devices or subsystems configured to acquire, generate, store, or transmit ECG signal data for analysis by system 1000. ECG device(s) 1020 may include, for example, a conventional 12-lead ECG machine, a bedside monitor, a telemetry monitor, a cart-based acquisition system, an ambulatory recorder, a patch monitor, a portable handheld ECG device, a wearable ECG device, a smartwatch-associated ECG sensor, or another device capable of measuring cardiac electrical activity and representing that activity in digital waveform form. In some embodiments, ECG device(s) 1020 may provide waveform samples from a full 12-lead configuration. In some embodiments, ECG device(s) 1020 may provide waveform samples from fewer than twelve leads, including one-lead, two-lead, three-lead, or other reduced-lead configurations. Accordingly, ECG device(s) 1020 may support both exemplary full-lead embodiments and reduced-lead or wearable embodiments of the disclosed techniques.

In some embodiments, ECG device(s) 1020 may transmit ECG signal data to server 1010 over network 1005 using a wired, wireless, or network-based connection. In some embodiments, ECG device(s) 1020 may provide raw waveform samples, median beat data, device-generated metadata, sampling rate information, calibration values, signal quality indicators, or other acquisition-related information that may be used by ECG ingestion module 1012 and downstream processing modules. In some embodiments, ECG device(s) 1020 may additionally receive outputs from server 1010, such as lead-level scores, ECG-level determinations, visual overlays, or alert signals for presentation on a device display or local interface. In some embodiments, at least part of the disclosed analysis may execute locally on ECG device(s) 1020, while other processing stages may execute remotely on server 1010. Thus, ECG device(s) 1020 may function as waveform-acquisition endpoints, partially intelligent edge devices, local visualization devices, or combinations thereof within the disclosed system architecture.

In some embodiments, user device(s) 1030 may include one or more computing devices through which a clinician, technician, reviewer, administrator, or other user may interact with system 1000. User device(s) 1030 may include desktop computers, laptop computers, tablet computers, smartphones, smart displays, portable clinical terminals, nurse-station workstations, telemetry review stations, emergency department consoles, cath-lab terminals, remote review terminals, or other network-connected computing devices. In some embodiments, user device(s) 1030 may provide an interface through which a user views ECG waveforms, lead-level scores, ECG-level determinations, alert outputs, serial trend information, or other outputs produced by HATW detection application 1011. In some embodiments, user device(s) 1030 may also be used to request analysis of stored ECG data, retrieve prior ECG studies, review comparison outputs, acknowledge alerts, or initiate downstream workflow actions.

In some embodiments, user device(s) 1030 may receive graphical outputs generated by output, alert, and visualization module 1018, including overlays of HATW scores on ECG waveforms, color-coded lead displays, per-lead component-score breakdowns, heatmaps, serial-comparison views, or culprit-localization indicators. In some embodiments, user device(s) 1030 may additionally transmit user inputs back to server 1010, such as requests for re-analysis, threshold-selection commands, display-setting adjustments, review notes, workflow acknowledgments, or case-routing actions. In some embodiments, user device(s) 1030 may be used in real-time clinical workflows, while in other embodiments they may be used for retrospective review, algorithm validation, training-data review, or administrative oversight. The label “user device(s)” is intended to be broad and non-limiting, and encompasses any endpoint through which a human or software-assisted user interacts with the outputs or control surfaces of the disclosed system.

In some embodiments, clinical system(s) 1040 may include one or more external healthcare, diagnostic, workflow, or enterprise systems that exchange information with server 1010 and HATW detection application 1011. Clinical system(s) 1040 may include, for example, electronic health record (EHR) systems, electronic medical record (EMR) systems, hospital information systems, order-entry systems, telemetry management systems, diagnostic workstations, cardiology review systems, triage systems, notification-routing platforms, or other clinical computing environments. In some embodiments, clinical system(s) 1040 may provide contextual information to server 1010, such as patient identifiers, encounter context, order status, historical ECG references, workflow state information, or routing instructions. In some embodiments, clinical system(s) 1040 may receive outputs generated by application 1011, including score results, alerts, workflow recommendations, serial trend information, or structured records indicating whether a given ECG satisfies one or more HATW-related criteria.

In some embodiments, communication between server 1010 and clinical system(s) 1040 may support integration of the disclosed HATW framework into broader clinical workflows. For example, when a threshold condition is satisfied, server 1010 may transmit an alert or structured result to an EHR system, a diagnostic workstation, a cath-lab triage system, or another clinical platform so that the HATW-related output may be presented, logged, escalated, or associated with the relevant patient encounter. In some embodiments, clinical system(s) 1040 may also provide serial ECG records, laboratory values, demographic data, or other contextual variables to support downstream pipeline integration, serial comparison, or composite diagnostic modeling. Thus, clinical system(s) 1040 may function as one or more integration endpoints through which HATW analysis is incorporated into existing clinical and enterprise infrastructure without requiring the disclosed techniques to remain isolated within a stand-alone scoring engine.

In some embodiments, database(s) 1050 may include one or more storage systems used by system 1000 to persist, retrieve, and manage waveform data, derived measurements, model information, threshold settings, and other records used throughout the disclosed workflows. Database(s) 1050 may be implemented using relational databases, document stores, object stores, waveform repositories, time-series databases, vector stores, file-based storage systems, or other storage technologies suitable for maintaining ECG-related data and associated processing state. In some embodiments, database(s) 1050 may store raw ECG recordings, processed waveform representations, median beats, segmentation results, lead eligibility data, morphology metrics, component scores, HATW scores, ECG-level determinations, serial-comparison data, culprit-localization outputs, visualization state information, alert records, model parameters, lead-group configurations, and audit or troubleshooting information. In some embodiments, database(s) 1050 may additionally store historical ECG studies for use in temporal comparison or calibration.

In some embodiments, one or more modules of HATW detection application 1011 may read from and write to database(s) 1050 throughout the analysis lifecycle. For example, ECG ingestion module 1012 may retrieve prior ECG studies or store newly received ECG objects; signal processing and waveform segmentation module 1013 may store processed waveform representations or segmentation boundaries; HATW scoring module 1016 may retrieve model parameters or coefficient sets; threshold and multi-lead evaluation module 1017 may retrieve deployment-specific threshold settings; output, alert, and visualization module 1018 may store rendered-output metadata or alert logs; serial comparison and culprit localization module 1019 may retrieve historical results for comparison; and pipeline interface module 1021 may store or retrieve export records and downstream integration status. In some embodiments, database(s) 1050 may be local to server 1010. In some embodiments, database(s) 1050 may be remote, distributed, replicated, or cloud-hosted. Thus, database(s) 1050 may provide the persistent data layer supporting execution continuity, serial analysis, workflow integration, model management, auditability, and deployment flexibility across the disclosed system.

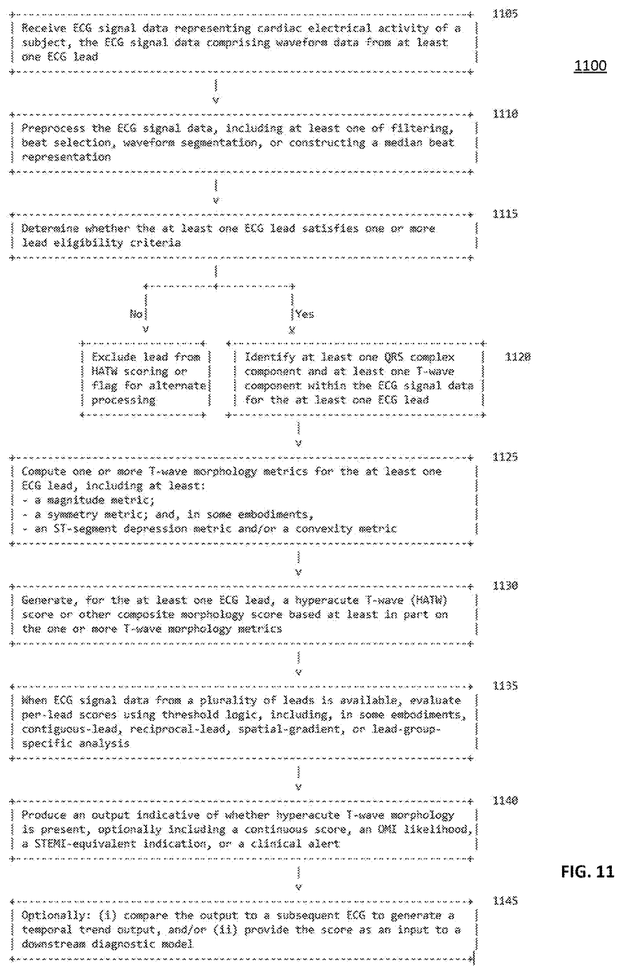

FIG. 11 illustrates an example method 1100 for identifying hyperacute T-waves in ECG data, in accordance with one or more embodiments. In various embodiments, method 1100 may be performed by one or more components of system 1000 shown in FIG. 10, including server 1010 executing HATW detection application 1011, although the method is not limited to that particular arrangement. Method 1100 provides a processor-executed workflow for receiving ECG signal data, preparing and evaluating one or more ECG leads, computing one or more T-wave morphology metrics, generating a HATW score or other composite morphology score, evaluating one or more threshold conditions, and producing one or more outputs indicative of hyperacute T-wave morphology. In some embodiments, method 1100 may be performed in real time or near real time as ECG data is acquired. In some embodiments, method 1100 may be performed on previously stored ECG studies, median beat representations, serial ECG recordings, or other waveform-derived data structures. Each step of method 1100 may correspond to operations performed by one or more modules of HATW detection application 1011, and may generate intermediate data used by downstream stages of the method.

At step 1105, one or more processors may receive ECG signal data representing cardiac electrical activity of a subject. In some embodiments, the received ECG signal data may comprise waveform data from at least one ECG lead and may further include waveform data from a plurality of ECG leads, such as a conventional 12-lead ECG acquisition or a reduced-lead acquisition generated by a portable or wearable device. The received ECG signal data may include raw waveform samples, filtered waveform samples, median beat data, previously segmented waveform data, or another digital representation of ECG morphology suitable for subsequent analysis. In some embodiments, the received data may also include associated metadata such as lead identifiers, acquisition timestamps, sample-rate information, calibration values, device identifiers, patient or study identifiers, signal-quality indicators, and one or more workflow-context fields indicating, for example, whether the ECG was acquired in an emergency department, telemetry, ambulatory, screening, or serial-monitoring setting.

In some embodiments, the ECG signal data may be received from ECG device(s) 1020 over network 1005, retrieved from database(s) 1050, provided by clinical system(s) 1040, or submitted through user device(s) 1030 for analysis by server 1010 executing HATW detection application 1011. In some embodiments, receiving the ECG signal data may include instantiating one or more internal waveform objects, associating the waveform objects with internal identifiers, and storing the waveform objects and associated metadata in memory for subsequent processing. In some embodiments, the processor may additionally determine whether the received ECG study should be linked to a prior ECG for the same subject, whether device-specific calibration or reduced-lead handling logic should be invoked, and whether the data should proceed through real-time analysis, deferred analysis, or serial-comparison processing. Thus, step 1105 may establish the waveform and context data structures used throughout the remainder of method 1100.

At step 1110, one or more processors may preprocess the ECG signal data to prepare one or more lead waveforms for morphology analysis. In some embodiments, preprocessing may include one or more signal-conditioning operations applied to the waveform data received at step 1105, such as filtering, baseline correction, denoising, beat alignment, beat selection, artifact attenuation, or another operation that improves the stability of later measurements. In some embodiments, preprocessing may additionally include identifying one or more candidate cardiac cycles within a recording, selecting one or more representative beats, and constructing a median beat representation for one or more leads. A median beat representation may reduce the effect of transient noise, beat-to-beat variability, or isolated acquisition artifacts and may provide a more stable waveform basis for delineating QRS and T-wave boundaries. In some embodiments, the processor may operate on a full waveform segment rather than a median beat, and the particular preprocessing operations used may vary depending on the source device, signal quality, rhythm characteristics, and deployment context.

In some embodiments, step 1110 may further include waveform segmentation operations that identify one or more ECG intervals or fiducial points used later in the method. For example, the processor may identify, for one or more leads, QRS onset, QRS peak, QRS offset, J-point location, T-wave onset region, T-wave peak, and T-wave end. In some embodiments, these delineation operations may be carried out using threshold-based logic, derivative-based routines, template matching, adaptive windows, model-assisted segmentation, or another processor-executed segmentation approach. The processor may store the resulting segmentation boundaries, confidence values, signal-quality indicators, and associated timing or amplitude information in one or more lead-level data structures for downstream use. Thus, step 1110 may convert ingested ECG waveform data into one or more processed waveform representations and segmentation records suitable for eligibility determination, morphology metric computation, and score generation later in method 1100.

At step 1120, one or more processors may identify one or more waveform components from the ECG signal data for use in subsequent morphology analysis. In some embodiments, the identified waveform components may include one or more QRS-related components, T-wave-related components, interval boundaries, fiducial points, extrema, segment locations, or other waveform features derived from the ECG signal data. This step may be performed using waveform data that has been preprocessed and, in some embodiments, qualified at earlier steps of method 1100. More generally, step 1120 may be understood as establishing the waveform-level information from which one or more morphology metrics, features, or model inputs are later derived.

In some embodiments, step 1120 may include identifying at least one QRS complex component and at least one T-wave component for a given lead. For example, one or more processors may identify one or more of: a QRS onset, a QRS offset, a QRS peak, a maximum QRS amplitude location, a minimum QRS amplitude location, a J-point, a T-wave onset region, a T-wave peak, and a T-wave end. In some embodiments, the processor may also determine one or more associated intervals, such as a QRS duration interval, a T-wave onset-to-peak interval, a T-wave peak-to-end interval, or another segment used in later computations. In some embodiments, the processor may retain more than one candidate boundary or fiducial point together with a confidence value, particularly where waveform definition is imperfect or where a later stage may benefit from uncertainty-aware processing.

In some embodiments, the waveform-component identification may be carried out using threshold-based routines, derivative-based routines, adaptive windowing, template matching, model-assisted segmentation, rule-based delineation, or another processor-executed technique for locating ECG waveform features. In some embodiments, the identified components may be stored in one or more lead-level data structures together with corresponding timing coordinates, sample indices, amplitude values, confidence indicators, or quality flags. These data structures may then be used directly in later steps to compute one or more morphology metrics, construct feature vectors, generate one or more scores, or support visualization, explanation, or auditability. Accordingly, step 1120 should be understood broadly as a waveform-component identification stage that prepares the ECG data for subsequent quantitative morphology analysis, rather than as being limited to any single delineation technique or set of component labels.

At step 1125, one or more processors may compute, derive, or otherwise obtain one or more morphology metrics associated with the ECG signal data. This step may use the waveform components identified at step 1120 and may transform those components into one or more quantitative descriptors informative of hyperacute T-wave morphology. In some embodiments, the one or more morphology metrics may include a first metric characterizing a relative size, extent, bulk, or amplitude-related property of a T wave with respect to one or more surrounding waveform features, and a second metric characterizing temporal symmetry, asymmetry, shape, or positional distribution of the T wave. In some embodiments, one or more additional metrics may also be computed, such as an ST-segment depression metric, a convexity metric, a reciprocal-lead metric, a spatial-gradient metric, an inter-lead metric, or another quantitative descriptor of waveform morphology. Thus, step 1125 may be understood broadly as a feature-generation stage in which one or more processors extract or derive quantitative morphology information from ECG data.

In some embodiments, the one or more morphology metrics may be computed directly from delineated waveform intervals and fiducial points. For example, one or more processors may determine amplitude values, time intervals, areas, slopes, curvature-related values, ratios, normalized quantities, transformed values, or other numerical measures using one or more QRS-related components and one or more T-wave-related components identified at step 1120. In some embodiments, the processor may compute a relative-size metric using a measure of T-wave area, T-wave amplitude, T-wave energy, or another T-wave extent measure normalized by a QRS-related quantity such as QRS amplitude, QRS area, QRS energy, QRS duration, or a composite thereof. In some embodiments, the processor may compute a temporal-morphology metric using a measure of peak location, onset-to-peak duration, peak-to-end duration, fractional peak position, slope relationship, skewness, area asymmetry, or another temporal characterization of the T wave. Accordingly, the specific metric definitions used at step 1125 may vary across embodiments, models, lead configurations, and deployment contexts.

In some embodiments, the morphology metrics computed at step 1125 may include the exemplary metrics used in the disclosed HATW framework. For example, a first metric may quantify relative T-wave size using information such as T-wave area and QRS amplitude, and a second metric may quantify temporal morphology using information such as the position of the T-wave peak within the T-wave interval. In some embodiments, these metrics may correspond to a T-wave magnitude metric and a T-wave symmetry metric. In some embodiments, one or more optional metrics may additionally be computed to capture other morphology patterns, including reciprocal changes, de Winter-type patterns, convex contour features, or multi-lead relationships. These exemplary metrics are illustrative only and should not be understood as limiting the broader metric-generation step.

In some embodiments, the one or more morphology metrics may be computed from a median beat representation, a selected beat, a multi-beat aggregate, or another waveform object derived from the ECG signal data. In some embodiments, one or more metrics may instead be computed from transformed representations of the waveform, including time-frequency representations, encoded representations, reduced-dimension feature sets, or other derived signal forms. In some embodiments, the metric set used for a given lead or ECG may vary according to lead group, acquisition type, device type, workflow setting, or model selection. In some embodiments, one or more processors may retain not only final metric values, but also one or more intermediate measurements used to derive those values, such as interval lengths, area values, amplitude values, curvature values, normalization values, or confidence indicators, so that those intermediate data can later be used for score explanation, visualization, audit, or serial comparison.

At step 1130, one or more processors may generate one or more scores, classifications, or other determinations based at least in part on the one or more morphology metrics obtained at step 1125. In some embodiments, the generated output may comprise a lead-level score associated with hyperacute T-wave morphology. In some embodiments, the generated output may comprise another composite morphology score, a pathology-indicative score, a confidence value, a binary determination, a multi-class determination, or another structured result derived from one or more morphology-related inputs. More generally, step 1130 may be understood as an inference or score-generation step in which one or more quantitative morphology descriptors are transformed into one or more outputs that indicate, estimate, classify, or otherwise characterize whether the ECG signal data exhibits hyperacute morphology or a related clinically meaningful pattern.

In some embodiments, the score-generation process may use a trained model. For example, one or more processors may apply a classification model, regression model, neural network, tree-based model, support vector machine, random forest, deep neural network, or another machine learning model to the morphology metrics obtained at step 1125. In some embodiments, the score-generation process may instead use rule-based logic, threshold-based logic, weighted combinations, lookup structures, or another deterministic or semi-deterministic scoring function. In some embodiments, the selected score-generation logic may depend on one or more attributes of the lead, recording, device type, workflow setting, or deployment configuration. In some embodiments, one or more processors may retrieve a selected model instance, coefficient set, or scoring configuration from memory before generating the score.

In some embodiments, the score generated at step 1130 may be based on explicitly computed morphology metrics, such as one or more metrics characterizing relative T-wave size and one or more metrics characterizing temporal T-wave morphology. In some embodiments, the score may be generated using a broader feature set that includes optional additional morphology descriptors, inter-lead features, reciprocal features, spatial features, transformed waveform features, or other ECG-derived inputs. In some embodiments, the score may be generated from learned waveform representations that do not correspond one-to-one to expressly named metrics, provided that the resulting output still reflects a processor-executed determination associated with hyperacute T-wave morphology. Accordingly, step 1130 is not limited to only one feature-to-score mapping, but instead encompasses a broader class of processor-executed scoring or classification operations using one or more morphology-related inputs.

In some embodiments, the score-generation process may produce one or more intermediate values in addition to a final score. For example, one or more processors may compute component scores, sub-scores, calibrated outputs, normalized outputs, probability-like values, or confidence indicators prior to producing a final score or classification result. In some embodiments, those intermediate values may be retained for later use in threshold evaluation, visualization, explanation, audit, model debugging, or downstream integration. In some embodiments, the final score may be represented as a continuous numerical value, such as a value on a bounded or unbounded scale, while in other embodiments the score may be converted immediately into one or more categorical or binary outputs. Step 1130 therefore serves as the principal stage in which morphology-derived information is converted into a structured result suitable for later interpretation, aggregation, and output generation.

In some embodiments, step 1130 may include generating the exemplary HATW score described elsewhere herein. For example, one or more processors may generate a score based at least in part on a T-wave magnitude metric and a T-wave symmetry metric and, in some embodiments, one or more optional additional metrics such as ST-segment depression or convexity. In some embodiments, different leads or lead groups may use different model instances, parameter sets, or scoring equations, such that the score-generation process is adapted to anatomical differences, waveform differences, or deployment-specific considerations. These exemplary implementations are illustrative only, and the broader step of generating one or more hyperacute-morphology-related outputs from one or more morphology metrics remains within the scope of step 1130.

At step 1135, one or more processors may evaluate one or more threshold conditions, aggregation conditions, or decision conditions based at least in part on the outputs generated at step 1130. In some embodiments, this evaluation may be performed on a per-lead basis. In some embodiments, this evaluation may be performed on a multi-lead basis, a per-ECG basis, a serial basis, or another aggregated basis. More generally, step 1135 may be understood as an interpretation stage in which one or more score outputs are analyzed to determine whether a selected condition for further action, classification, routing, or output generation has been satisfied.

In some embodiments, step 1135 may include organizing one or more lead-level scores according to anatomical adjacency, lead-group membership, reciprocal relationships, spatial relationships, temporal relationships, or another structural arrangement and then applying one or more decision rules to the organized set. For example, one or more processors may determine whether one or more leads, groups of leads, or combinations of leads satisfy a threshold condition, whether a mean or other aggregate of multiple scores satisfies a selected criterion, whether a required number of leads exceed a selected value, or whether one or more reciprocal or corroborating morphology patterns are present. In some embodiments, the decision condition may be based on contiguous leads. In some embodiments, the condition may instead or additionally depend on non-contiguous leads, lead-group-specific patterns, inter-lead metric relationships, reciprocal-lead patterns, spatial gradients, or other cross-lead relationships. Accordingly, step 1135 is not limited to a single threshold rule, but instead encompasses broader ECG-level interpretation of one or more lead-level or segment-level outputs.

In some embodiments, the threshold or decision logic may be configurable and context-dependent. Different threshold values, aggregation modes, adjacency rules, positivity criteria, or routing criteria may be used in different deployment settings. For example, one configuration may favor high specificity for alert generation or STEMI-equivalent use, while another configuration may favor greater sensitivity for screening, monitoring, or serial review. In some embodiments, the selected rule may depend on whether the ECG data was acquired from a full 12-lead system, a reduced-lead system, a wearable device, or another source. In some embodiments, one or more processors may also evaluate whether enough suitable leads or score outputs are available to support an ECG-level determination and may generate an indeterminate or insufficient-data result when appropriate.

In some embodiments, step 1135 may include applying the exemplary multi-lead positivity logic described elsewhere herein. For example, one or more processors may determine whether at least two contiguous leads satisfy a selected threshold condition, such as whether a mean of two contiguous lead-level scores meets or exceeds a selected value. In some embodiments, other threshold values, including more permissive or more restrictive values, may be used. In some embodiments, the selected logic may be adapted to lead-group-specific scoring, reduced-lead configurations, or workflow-specific operating conditions. These exemplary implementations are illustrative only, and the broader evaluation of one or more threshold or aggregation conditions based on one or more morphology-related outputs remains within the scope of step 1135.

At step 1140, one or more processors may generate and output one or more results associated with the analysis performed in method 1100. In some embodiments, the output may comprise a lead-level output, an ECG-level output, or both. The output may include, for example, one or more scores, a positive or negative determination, a classification result, a confidence indicator, a pathology-likelihood indicator, a threshold-satisfaction indicator, a component-score breakdown, or another structured result derived from the earlier steps of the method. More generally, step 1140 may be understood as the stage at which one or more processors translate morphology analysis and score interpretation into one or more outputs suitable for use by users, systems, workflows, or downstream computational processes.

In some embodiments, the output generated at step 1140 may be formatted for presentation to a user. For example, one or more processors may generate a display-oriented output in which one or more scores, classifications, or indicators are overlaid on or presented adjacent to one or more ECG waveforms, lead labels, heatmaps, lead arrays, or other visual structures. In some embodiments, one or more visual differentiation techniques may be used, such as color coding, highlighting, flagging, iconography, or other display cues associated with threshold status, severity, confidence, or lead contribution. In some embodiments, the user-facing output may further include one or more component values, sub-scores, intermediate measurements, or explanatory data indicating how a particular lead-level or ECG-level result was produced.

In some embodiments, the output generated at step 1140 may be machine-readable and may be transmitted, stored, queued, or otherwise made available to one or more external systems. For example, the output may be represented as a structured message, data object, record, API payload, notification object, workflow trigger, or stored result. In some embodiments, the output may include one or more identifiers, timestamps, model identifiers, threshold settings, configuration values, quality indicators, or provenance information so that the result may be interpreted correctly by a downstream recipient. In some embodiments, both a user-facing output and a machine-readable output may be generated from the same underlying analysis result.

In some embodiments, step 1140 may include generating an alert or other escalatory output when one or more selected conditions are satisfied. Such an alert may indicate that the ECG data exhibits hyperacute T-wave morphology, may indicate a likelihood of occlusion myocardial infarction, may identify the ECG as exhibiting a STEMI-equivalent pattern, or may otherwise designate the result as clinically actionable. In some embodiments, the alert may recommend or support one or more actions, such as expedited review, emergent reperfusion evaluation, catheterization evaluation, serial monitoring, or another workflow pathway. In some embodiments, the output may additionally include supporting information such as the lead or leads contributing to the result, one or more component values, one or more temporal trend values, or one or more localization-related outputs. Thus, step 1140 encompasses a broad class of output-generation behaviors rather than only one alert format or one display format.

At step 1145, one or more processors may optionally perform one or more additional operations using the outputs generated at step 1140. In some embodiments, this step may include comparing a current result to one or more prior or subsequent ECG-related results to determine whether a temporal change is present. For example, one or more processors may receive or access one or more additional ECG recordings for the same subject and may compare one or more current scores, classifications, or morphology-related values to corresponding values from the other ECG recordings. In some embodiments, the resulting comparison may indicate new appearance of hyperacute morphology, progression, improvement, resolution, recurrence, or another temporal trend. In some embodiments, one or more processors may generate a temporal trend output, a serial comparison output, or an alert based at least in part on such a comparison. Thus, in some embodiments, step 1145 may support serial monitoring and dynamic interpretation of changes over time rather than only one-time analysis of an isolated ECG recording.

In some embodiments, step 1145 may additionally or alternatively include providing one or more outputs from method 1100 to a downstream system, model, workflow, or data-processing pipeline. For example, one or more scores, labels, classifications, confidence indicators, or related outputs may be transmitted to a downstream diagnostic model, an automated ECG interpretation system, a workflow engine, a storage system, an analytics platform, or a model-training pipeline. In some embodiments, such outputs may be used as input features, training targets, supervisory signals, routing indicators, or historical reference values. In some embodiments, a downstream model may combine one or more outputs of method 1100 with one or more additional ECG-derived, demographic, laboratory, or clinical inputs to generate another diagnostic or prognostic output. Accordingly, step 1145 broadly captures optional serial use, integrative use, and downstream use of the results generated by the core hyperacute morphology analysis method.

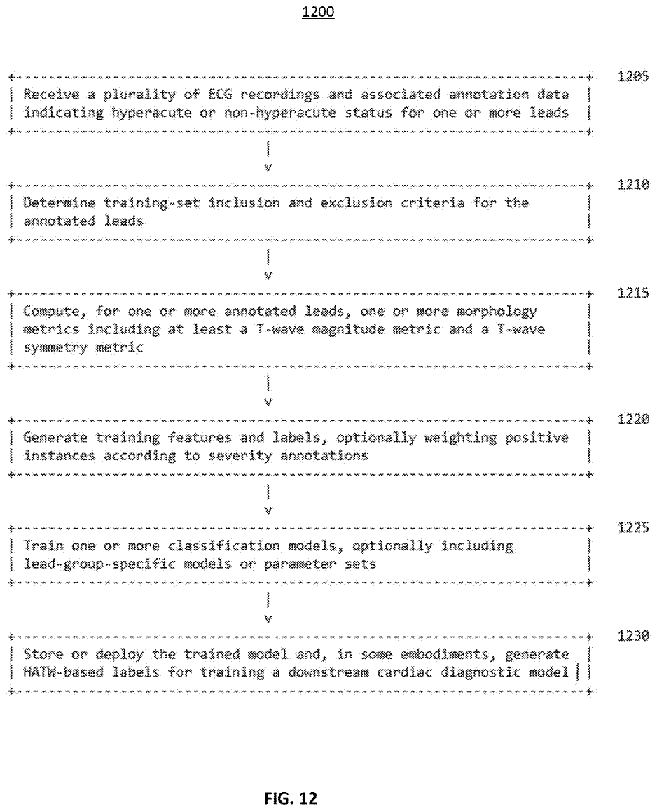

FIG. 12 illustrates an example method 1200 for training a hyperacute T-wave classification model and, in some embodiments, for generating one or more model artifacts, supervisory signals, or downstream training outputs derived from ECG data. Whereas FIG. 11 describes an example inference-time workflow for analyzing ECG data and generating one or more hyperacute-morphology-related outputs for a given ECG study, FIG. 12 describes an example development- and training-time workflow in which one or more processors use ECG recordings, annotation data, and one or more derived waveform representations or morphology metrics to train, calibrate, configure, validate, or otherwise prepare one or more models for later deployment. In various embodiments, method 1200 may be performed by one or more components of ECG system 1000 of FIG. 10, including server 1010 executing HATW detection application 1011, although the method is not limited to that arrangement. The steps shown in FIG. 12 are illustrative and, in some embodiments, may be performed in different orders, combined, subdivided, repeated, or omitted depending on the training architecture, annotation scheme, feature set, validation strategy, or downstream use being implemented.

At step 1205, one or more processors may receive a plurality of ECG recordings and associated annotation data for use in training one or more hyperacute T-wave-related models. In some embodiments, the ECG recordings may include full 12-lead ECG studies, reduced-lead recordings, median beat representations, segmented waveform objects, or other ECG-derived data structures suitable for training-time processing. The associated annotation data may include binary labels, categorical labels, severity values, lead-level markings, ECG-level labels, region annotations, or other supervisory information indicating whether one or more portions of the ECG data correspond to hyperacute or non-hyperacute morphology. In some embodiments, the annotation data may be provided by one or more expert reviewers, adjudicators, or other annotation workflows. In some embodiments, the annotation data may additionally include contextual information such as study identifiers, lead identifiers, outcome-linked labels, quality indicators, or annotation-confidence information. Thus, step 1205 may establish the training corpus and associated supervisory inputs used throughout method 1200.

In some embodiments, the ECG recordings and annotation data received at step 1205 may come from different sources and may require harmonization before training-time use. For example, one or more processors may receive ECG recordings from one or more sites, devices, repositories, or historical data stores and may receive annotations from one or more review tools, spreadsheets, structured records, databases, or labeled waveform objects. In some embodiments, the processor may associate each ECG recording with one or more lead-level and/or study-level annotation records, may resolve differences in labeling conventions, may normalize metadata fields, and may instantiate one or more internal training examples, lead objects, or study objects for subsequent preparation. In some embodiments, the received training corpus may include recordings later allocated among derivation, calibration, tuning, validation, or test subsets. Step 1205 is therefore not limited to one ingestion format or one supervision scheme, but instead broadly encompasses receipt of ECG data and associated supervisory information for model training and related training-time operations.

At step 1210, one or more processors may determine training-set inclusion and exclusion criteria for one or more ECG recordings, leads, waveform segments, or annotation instances. In some embodiments, this step may include selecting which portions of the received corpus should be used as positive examples, which portions should be used as negative examples, which portions should be withheld, and which portions should be excluded altogether from one or more training phases. The inclusion or exclusion logic may depend on annotation status, waveform quality, eligibility conditions, label confidence, study configuration, deployment target, or another training-related criterion. In some embodiments, the processor may generate one or more inclusion flags, exclusion flags, quality indicators, or weighting indicators associated with the training examples. This step may therefore function as a curation stage in which one or more processors prepare a training set intended to reflect the morphology distinctions relevant to the model being trained.

In some embodiments, the inclusion or exclusion logic may be designed to align the training process with a selected deployment use case. For example, one or more processors may exclude leads or recordings associated with particular ECG categories, may exclude poorly defined or ineligible waveforms, may exclude training negatives that could be contaminated by hyperacute morphology elsewhere in the same recording, or may otherwise limit the training set to examples deemed appropriate for the intended scoring framework. In some embodiments, training examples associated with one or more selected ECG categories, including STEMI-positive cases, may be excluded from one or more training phases to emphasize performance in a selected target population. In some embodiments, the exclusion logic may instead be relaxed or modified for broader model-development embodiments. Thus, step 1210 encompasses a broad range of processor-executed training-set curation operations, including filtering, partitioning, qualification, and contamination-reduction logic.

At step 1215, one or more processors may compute, derive, or otherwise obtain one or more morphology metrics or other training features from one or more ECG recordings selected for model training. In some embodiments, the training features may include one or more features of the kind described with respect to FIG. 11, such as one or more metrics characterizing relative T-wave size, temporal symmetry or asymmetry, ST-segment behavior, convexity, reciprocal relationships, inter-lead relationships, spatial gradients, transformed waveform characteristics, or other quantitative morphology descriptors. In some embodiments, the training features may be computed on a lead-level basis. In some embodiments, the training features may be computed on an ECG-level basis or on another basis such as grouped-lead, segment-level, or multi-lead feature-vector basis. More generally, step 1215 may be understood as a feature-generation stage in which one or more processors transform one or more training ECG recordings into one or more structured inputs suitable for model fitting.

In some embodiments, the features computed at step 1215 may include the exemplary T-wave magnitude and T-wave symmetry metrics used in the disclosed HATW framework. In some embodiments, one or more processors may derive those metrics from delineated waveform components, median beat representations, or other processed waveform structures. In some embodiments, one or more optional metrics, such as ST-segment depression or convexity, may also be computed. In some embodiments, the feature set may vary depending on the intended model family, lead group, device type, or deployment target. In some embodiments, the processor may generate explicit numerical feature vectors for use by one or more classical machine learning models. In some embodiments, the processor may instead prepare waveform-derived tensors, encoded waveform objects, or other representations suitable for training one or more deep learning models. Accordingly, step 1215 is not limited to one fixed feature set, but instead encompasses broader training-time derivation of one or more inputs informative of hyperacute morphology.

At step 1220, one or more processors may generate training labels, training weights, or other supervisory values associated with the features or examples prepared for training. In some embodiments, this step may include converting annotation data into one or more machine-usable supervisory structures, such as binary labels, categorical labels, severity-weighted labels, class-probability targets, confidence-weighted targets, or other representations suitable for model fitting. In some embodiments, a positive example may be associated with a severity value or another rating that affects its training weight. In some embodiments, negative examples may be assigned a default weight, a context-dependent weight, or another weighting value. In some embodiments, one or more processors may generate labels on a lead-by-lead basis, on an ECG-by-ECG basis, or on another analysis basis consistent with the model being trained. Step 1220 may therefore function as the supervision-construction stage of method 1200.

In some embodiments, the supervisory information generated at step 1220 may be used not only for training a HATW classification model, but also for one or more downstream or related training purposes. For example, one or more processors may generate one or more labels or target structures that reflect whether a lead, recording, or grouped lead pattern satisfies one or more HATW-related conditions. In some embodiments, those labels may later be used as training targets for a downstream cardiac diagnostic model, a raw-waveform model, an automated interpretation system, or another machine learning system that incorporates or learns from the disclosed HATW framework. In some embodiments, one or more processors may also associate one or more labels with one or more quality or ambiguity indicators so that uncertain or borderline examples can be weighted, reviewed, or handled differently during training. Thus, step 1220 broadly encompasses creation and structuring of supervisory signals for one or more training-time uses.

At step 1225, one or more processors may train one or more classification models, scoring models, or related predictive models using the training inputs and supervisory information prepared in earlier steps. In some embodiments, the model being trained may be configured to generate one or more outputs indicative of hyperacute T-wave morphology. In some embodiments, the model may be a lead-level model. In some embodiments, the model may be an ECG-level model, a grouped-lead model, or another predictive structure that consumes one or more morphology-related inputs and produces one or more HATW-related outputs. More generally, step 1225 may be understood as the stage in which one or more processors fit, estimate, optimize, calibrate, or otherwise configure one or more models using ECG-derived training data and associated supervision.

In some embodiments, the trained model may comprise logistic regression, a feedforward neural network, a tree-based model, a support vector machine, a random forest, a deep neural network, or another machine learning model. In some embodiments, the processor may train a model using explicitly computed morphology metrics such as relative-size and temporal-morphology features. In some embodiments, the processor may train separate models or separate parameter sets for different lead groups, anatomical regions, device configurations, or deployment settings. In some embodiments, the processor may optimize one or more training objectives using weighted examples, regularization, calibration procedures, cross-validation, hyperparameter tuning, or other model-development techniques. In some embodiments, the processor may additionally validate, score, or compare one or more candidate models on one or more holdout sets or validation subsets before selecting a model for deployment. Accordingly, step 1225 is not limited to any one training algorithm or architecture, but instead encompasses broader processor-executed training and model-selection operations for generating one or more HATW-related models.

At step 1230, one or more processors may store, deploy, export, or otherwise make available one or more results of the training process. In some embodiments, the resulting artifacts may include one or more trained models, parameter sets, coefficient sets, calibration parameters, threshold settings, validation outputs, model identifiers, feature definitions, label definitions, or associated metadata. In some embodiments, one or more processors may store the trained model for later use in inference-time workflows such as the method of FIG. 11. In some embodiments, one or more processors may additionally export one or more HATW-related labels, scores, targets, or other supervisory artifacts for downstream model training, analytics, or integration into broader diagnostic systems. Step 1230 may therefore function as the deployment and artifact-output stage of method 1200.

In some embodiments, the outputs of step 1230 may be used in multiple ways. For example, one or more trained models may be installed, registered, or referenced by HATW detection application 1011 for later runtime scoring, while one or more thresholded HATW labels or score-derived outputs may be provided to a downstream cardiac diagnostic model as training targets or input features. In some embodiments, the processor may preserve additional provenance information, such as training data version, lead-group configuration, feature selection, model family, threshold configuration, validation summary, or deployment status, so that later inference, audit, retraining, or comparison operations can be performed consistently. Thus, step 1230 broadly captures post-training handling of model artifacts and related outputs, including deployment of a HATW model and generation of one or more downstream training or integration products.

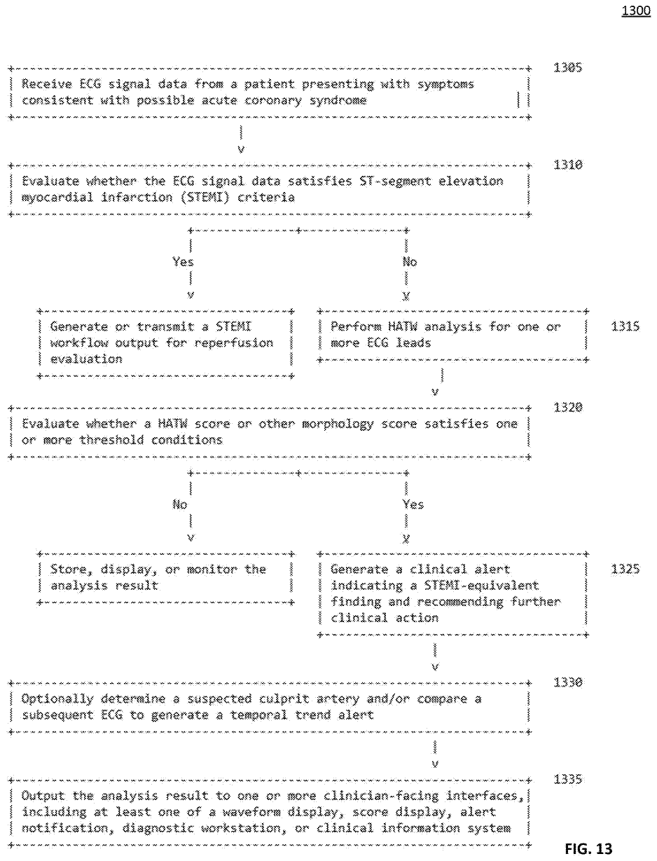

FIG. 13 illustrates an example method 1300 for integrating hyperacute T-wave analysis into a clinical decision-making workflow. Whereas FIG. 11 describes an example analysis-time workflow for generating one or more hyperacute-morphology-related outputs from ECG data, and FIG. 12 describes an example training-time workflow for preparing one or more models used in such analysis, FIG. 13 describes an example use-time workflow in which one or more processors incorporate HATW-related analysis into a broader evaluation pathway associated with suspected acute coronary syndrome, reperfusion decision support, serial review, and clinical communication. In various embodiments, method 1300 may be performed by one or more components of ECG system 1000 of FIG. 10, including server 1010 executing HATW detection application 1011, although the method is not limited to that arrangement. The steps shown in FIG. 13 are illustrative and, in some embodiments, may be performed in different orders, combined, subdivided, repeated, or omitted depending on the deployment environment, patient workflow, alerting logic, or clinical integration pathway being used.

At step 1305, one or more processors may receive ECG signal data associated with a subject undergoing evaluation in a clinical workflow. In some embodiments, the subject may be a patient presenting with symptoms consistent with possible acute coronary syndrome, chest pain, ischemia, myocardial infarction, or another clinical condition for which ECG evaluation is relevant. In some embodiments, the ECG signal data may be received directly from an acquisition device, from a monitoring system, from a storage system, from a clinical information system, or from another component of ECG system 1000. In some embodiments, the received data may be associated with a single ECG study, while in other embodiments the received data may be part of a serial monitoring sequence, a repeat-ECG workflow, a triage pathway, or another ongoing clinical evaluation context. Step 1305 therefore establishes the clinical-use context in which the HATW-related analysis is to be applied.

At step 1310, one or more processors may evaluate whether the ECG signal data satisfies one or more STEMI-related criteria or another initial gating condition used in a clinical workflow. In some embodiments, this evaluation may be performed using one or more preexisting ECG analysis processes, one or more processor-executed rules, one or more stored criteria, or another gating mechanism that determines whether the case is immediately routed into a first workflow branch or instead proceeds to additional HATW-related analysis. In some embodiments, the STEMI-related evaluation may generate a positive result, a negative result, an indeterminate result, or another routing indicator. More generally, step 1310 may serve as an initial workflow gate used to determine how one or more processors should handle the ECG within the surrounding clinical pathway.

In some embodiments, if the ECG signal data satisfies the initial gating condition, one or more processors may route the case into a workflow branch associated with immediate or expedited clinical handling, while still optionally preserving the ECG for later HATW-related analysis, confirmatory analysis, archival purposes, or explanatory display. In some embodiments, if the ECG signal data does not satisfy the initial gating condition, one or more processors may route the case into a branch in which HATW analysis is performed to identify one or more clinically significant patterns that may not be captured by the initial gate. Accordingly, step 1310 should be understood broadly as a workflow-routing step rather than being limited to one particular clinical rule set.

At step 1315, one or more processors may perform HATW-related analysis for one or more ECG leads. In some embodiments, this step may include carrying out all or part of the method of FIG. 11, including generating one or more morphology metrics, scores, classifications, or other outputs indicative of hyperacute T-wave morphology. In some embodiments, the analysis may be performed only when the ECG signal data does not satisfy the initial gate evaluated at step 1310. In some embodiments, the analysis may instead be performed regardless of the outcome of step 1310, such as to provide confirmatory information, secondary classification information, serial-comparison support, or additional workflow context. Step 1315 may therefore operate as the stage at which one or more HATW-related outputs are generated for use in subsequent clinical workflow decisions.

At step 1320, one or more processors may evaluate whether one or more outputs produced at step 1315 satisfy one or more workflow decision criteria. In some embodiments, the decision criteria may include one or more threshold conditions, score conditions, pattern-recognition conditions, aggregation rules, temporal conditions, or routing rules. For example, one or more processors may determine whether a HATW score, composite morphology score, or ECG-level result exceeds a selected threshold, whether a particular lead pattern is present, whether multiple leads satisfy one or more conditions, or whether one or more serial changes are sufficiently significant to warrant further action. In some embodiments, the selected decision criteria may be configured for a particular workflow setting, such as emergency department triage, serial monitoring, pre-hospital screening, specialist review, or another use environment. Step 1320 may therefore function as the clinical-decision interpretation stage of method 1300.

At step 1325, one or more processors may generate one or more workflow-related outputs based on the evaluation performed at step 1320. In some embodiments, if one or more selected conditions are satisfied, the processor may generate an alert, a recommendation, a workflow escalation output, or another clinically actionable result. Such an output may indicate that the ECG exhibits hyperacute T-wave morphology, may identify the ECG as exhibiting a STEMI-equivalent pattern, may indicate a likelihood of occlusion myocardial infarction, or may otherwise classify the case as requiring expedited attention. In some embodiments, the output may recommend or support one or more actions, such as emergent catheterization evaluation, emergent reperfusion evaluation, expedited cardiology consultation, repeat ECG acquisition, serial monitoring, or another workflow branch. In some embodiments, if the selected conditions are not satisfied, the processor may instead generate an output indicating continued monitoring, storage, standard workup, deferred review, or another non-escalated workflow state. Thus, step 1325 encompasses both escalatory and non-escalatory workflow outputs generated using HATW-related analysis.

In some embodiments, the workflow-related output may include more than a single determination. For example, one or more processors may include a lead-level score breakdown, one or more component metrics, a confidence indicator, a temporal trend indication, or one or more supporting data elements that explain or contextualize the workflow result. In some embodiments, the output may be formatted for a user-facing clinical interface, a machine-readable workflow engine, a clinical information system, a mobile notification channel, a bedside monitor, or another communication pathway. Accordingly, step 1325 should be understood broadly as a processor-executed generation of one or more clinical workflow outputs based at least in part on hyperacute-morphology-related analysis.

At step 1330, one or more processors may optionally generate one or more additional interpretation outputs associated with spatial distribution or temporal change. In some embodiments, this step may include determining a suspected culprit artery or anatomical region based at least in part on the distribution of elevated scores, positive leads, reciprocal changes, lead-group patterns, or other spatial relationships among the ECG leads. In some embodiments, this step may additionally or alternatively include receiving one or more subsequent ECG recordings for the same subject and generating a temporal trend output based on changes in one or more HATW-related results across time. In some embodiments, the temporal trend output may indicate new appearance, progression, persistence, improvement, or resolution of one or more morphology-related patterns. Thus, step 1330 may extend the workflow beyond a single static analysis result and may provide one or more higher-level interpretation outputs useful in clinical decision making.

At step 1335, one or more processors may communicate, display, transmit, store, or otherwise provide the workflow output to one or more downstream recipients or systems. In some embodiments, the output may be provided to a clinician-facing interface, a diagnostic workstation, a bedside monitor, a mobile device, an electronic health record system, a hospital information system, a telemetry management platform, a workflow engine, or another software or hardware endpoint. In some embodiments, the provided output may include a visual representation of one or more ECG waveforms together with one or more lead-level or ECG-level results. In some embodiments, the provided output may instead or additionally include one or more structured records, alerts, notifications, routing instructions, audit entries, or workflow-state updates. Step 1335 may therefore function as the communication and integration stage of method 1300, at which one or more results of the HATW-integrated workflow are delivered for use in the surrounding clinical environment.

In some embodiments, the communications performed at step 1335 may support both immediate clinical action and later review. For example, one or more processors may transmit an alert for urgent review while also storing one or more associated scores, supporting values, timestamps, and routing results for audit, quality analysis, serial comparison, downstream modeling, or retrospective workflow evaluation. In some embodiments, the communicated output may include one or more explanatory data elements, such as the leads contributing to a positive result, the threshold logic satisfied, one or more trend values, or one or more localization-related outputs. Accordingly, step 1335 broadly encompasses communication and persistence of one or more clinically integrated HATW-related workflow results, rather than being limited to only one display mode or one notification channel.

In some embodiments, the disclosed techniques are not limited to only the particular exemplary magnitude and symmetry formulations described elsewhere herein. More generally, one or more processors may derive a first morphology metric characterizing a relative size, extent, bulk, amplitude-related property, or energy-related property of a T wave with respect to one or more surrounding waveform features, and may derive a second morphology metric characterizing temporal symmetry, asymmetry, shape, peak distribution, or positional distribution of the T wave. One or more processors may then combine those metrics to generate a composite morphology score indicative of hyperacute morphology. In some embodiments, the first morphology metric may correspond to a T-wave magnitude metric and the second morphology metric may correspond to a T-wave symmetry metric. In some embodiments, the first and second metrics may instead be implemented using alternative quantitative formulations that still characterize relative T-wave size and temporal T-wave morphology. Accordingly, the disclosed HATW score may be understood as an exemplary implementation within a broader processor-executed framework for generating a composite morphology score from one or more quantitative T-wave descriptors.

In some embodiments, the first morphology metric may be computed using one or more measures selected from T-wave area, T-wave amplitude, T-wave energy, root-mean-square T-wave amplitude, integrated T-wave magnitude over one or more intervals, or another measure of T-wave extent, optionally normalized using one or more QRS-related quantities such as QRS amplitude, QRS area, QRS energy, QRS duration, or a composite thereof. In some embodiments, the second morphology metric may be computed using one or more measures selected from peak-position ratios, onset-to-peak and peak-to-end relationships, skewness, slope comparisons, area asymmetry, curvature-related features, or other temporal or geometric characterizations of the T wave. In some embodiments, one or more processors may apply monotonic transformations, normalizations, scalings, calibrations, or other mathematical operations to such quantities before they are used for score generation. Thus, the disclosed techniques are not limited to only one exact pair of mathematical expressions even where specific examples are discussed elsewhere herein.

In some embodiments, one or more additional morphology descriptors may be incorporated into the analysis in addition to, or in place of, one or more of the exemplary metrics. Such additional descriptors may include an ST-segment depression metric, a convexity metric, one or more slope-related measures, one or more reciprocal-lead measures, one or more multi-lead or spatial-gradient measures, one or more transformed waveform features, or one or more features generated from time-frequency analysis, dimensionality reduction, encoded waveform representations, or other signal-derived transformations. In some embodiments, one or more processors may compute any subset of the disclosed features for use in score generation. In some embodiments, different feature subsets may be used for different lead groups, different device types, different patient populations, different deployment settings, or different model architectures. The disclosed techniques therefore encompass not only the exemplary two-feature implementation, but also broader feature sets tailored to particular analysis goals or deployment environments.

In some embodiments, one or more processors may generate a HATW-related score or classification using a variety of model families. In some embodiments, the score-generation logic may comprise logistic regression, a feedforward neural network, a gradient-boosted decision tree model, a support vector machine, a random forest, a deep neural network, or another machine learning model. In some embodiments, the score-generation logic may instead comprise a rule-based or semi-rule-based approach using one or more thresholds, weighted combinations, Boolean conditions, score tables, or other deterministic logic. In some embodiments, one or more processors may select among multiple available models based on lead group, lead configuration, device type, workflow setting, or a deployment profile stored in memory. Accordingly, the disclosure is not limited to only one model architecture, even though particular exemplary scoring models may be described in detail elsewhere herein.

In some embodiments, one or more processors may train or use models that rely on explicitly computed morphology metrics. In some embodiments, one or more processors may instead train or use models that consume waveform-derived inputs and learn internal representations corresponding to morphology patterns without explicitly computing all named metrics at inference time. For example, one or more processors may train a convolutional neural network, recurrent neural network, long short-term memory network, transformer-based model, hybrid neural architecture, or another deep model to receive raw waveform data, preprocessed waveform data, median beat data, or encoded waveform objects and to produce one or more HATW-related outputs. In some embodiments, such a model may be trained using labels, supervision signals, score-derived targets, or outcome-linked targets associated with the disclosed HATW framework. Thus, the present disclosure encompasses both explicit-metric embodiments and learned-representation embodiments.

In some embodiments, the disclosed techniques may use lead-group-specific parameterization. For example, one or more processors may divide ECG leads into multiple lead groups, such as limb leads, right precordial leads, left precordial leads, other anatomical groupings, empirically derived clusters, configurable lead sets, or even lead-by-lead groupings. In some embodiments, each group may have its own model instance, coefficient set, calibration parameters, thresholds, or feature definitions. In some embodiments, the relative contribution of one or more morphology metrics to a score may differ by lead group or clinical context. In some embodiments, a lead-group configuration may be predetermined, learned, configurable, or generated dynamically from training data. Accordingly, the lead-group-specific embodiments described herein are illustrative and need not be limited to only one grouping arrangement.

In some embodiments, reduced-lead and wearable-device implementations may be used. For example, the ECG signal data may comprise fewer than twelve leads, including one lead, two leads, three leads, six leads, or another subset of leads generated by a portable or wearable device. In some embodiments, one or more processors may apply recalibrated score-generation logic, modified feature definitions, alternative lead-group arrangements, or device-specific training parameters when analyzing reduced-lead ECG data. In some embodiments, one or more processors may generate a reduced-lead screening score configured to identify cases that should be escalated to full 12-lead acquisition or specialist review. In some embodiments, the threshold logic used for reduced-lead or wearable embodiments may differ from the threshold logic used for full 12-lead ECG studies. Thus, the disclosed techniques may be deployed across conventional clinical ECG systems and lower-lead or consumer-oriented ECG devices alike.

In some embodiments, the outputs generated by the disclosed techniques may be visualized in various ways. For example, one or more processors may generate waveform overlays showing one or more lead-level scores adjacent to or superimposed on ECG waveforms, color-coded threshold indicators, one or more component-score values, lead-array visualizations, heatmaps, decision-boundary views, serial trend displays, or other visual representations of the analysis results. In some embodiments, visualization outputs may include one or more values associated with magnitude-related features, symmetry-related features, ST-depression-related features, convexity-related features, or confidence indicators. In some embodiments, the visualization may be configured for rapid clinical interpretation, retrospective review, model debugging, training-data review, or system validation. These visualization embodiments may be implemented on user devices, clinical workstations, ECG devices, remote review systems, or other display-capable endpoints.

In some embodiments, the disclosed techniques may be integrated into one or more downstream computational pipelines. For example, one or more processors may provide one or more HATW-related scores, classifications, thresholded outputs, or derived labels to a downstream diagnostic model, an automated ECG interpretation system, a workflow engine, an analytics platform, a triage engine, or another software component. In some embodiments, a downstream model may combine one or more HATW-related outputs with one or more additional ECG-derived, demographic, laboratory, hemodynamic, or clinical features to generate a composite diagnostic output. In some embodiments, one or more HATW-related outputs may be used as input features for a broader OMI-detection model, ischemia model, cardiac-risk model, or rule-based decision-support system. Thus, the disclosed HATW framework may function as a stand-alone analysis system or as a feature-generating subsystem within a broader clinical or computational architecture.

In some embodiments, one or more HATW-related outputs may be used as training targets or supervisory signals for downstream machine learning systems. For example, one or more processors may generate lead-level or ECG-level HATW labels from one or more scores, thresholds, or expert-associated supervision signals and may use those labels to train a downstream model that operates on raw waveform data or other ECG-derived inputs. In some embodiments, such a downstream model may produce one or more outputs indicative of hyperacute morphology, occlusion myocardial infarction, ischemia, or another cardiac condition without explicitly computing all morphology metrics at inference time. In some embodiments, the disclosed HATW-related labels or score-derived outputs may enable large-scale training of downstream models even where direct expert annotation capacity is limited. Accordingly, the present disclosure encompasses use of the disclosed scoring framework both for direct analysis and for generating supervisory structures for broader model development.

In some embodiments, temporal analysis may be performed across multiple ECG recordings for the same subject. One or more processors may compare one or more current scores, classifications, or morphology metrics to corresponding values from one or more earlier or later ECGs and may determine whether the relevant morphology is newly appearing, increasing, decreasing, persisting, or resolving. In some embodiments, one or more processors may generate one or more temporal trend outputs, serial comparison outputs, or temporal alerts based at least in part on such comparisons. In some embodiments, the temporal analysis may use lead-level comparisons, ECG-level comparisons, aggregated comparisons, or multi-timepoint trajectory analysis. In some embodiments, the temporal outputs may be provided to clinicians, workflow systems, downstream diagnostic models, or analytics systems. Thus, the disclosed techniques are not limited to one-time analysis of isolated ECGs, but may also support serial monitoring and time-dependent interpretation.

In some embodiments, the disclosed techniques may be used to generate a variety of output formats beyond a single score or binary result. For example, one or more processors may generate per-lead scores, per-ECG determinations, confidence indicators, OMI-likelihood outputs, STEMI-equivalent indicators, alert recommendations, culprit-localization outputs, temporal comparison outputs, component-score breakdowns, or one or more structured data objects combining multiple such results. In some embodiments, different output formats may be generated for different recipients, such as clinicians, automated systems, EHR systems, diagnostic workstations, or model-training pipelines. In some embodiments, the output format may be configured dynamically based on workflow context, user role, device capability, or deployment profile. Accordingly, the disclosed techniques encompass a broad family of output behaviors, not merely a single displayed score value.

In some embodiments, one or more workflow conditions, thresholds, or routing criteria used by the disclosed techniques may be configurable. For example, one or more processors may store one or more deployment profiles specifying selected thresholds, model versions, lead-group definitions, alerting rules, visualization formats, reduced-lead handling logic, serial-comparison logic, or downstream integration rules. In some embodiments, a selected deployment profile may emphasize specificity, sensitivity, screening performance, high-acuity triage, automated interpretation, wearable monitoring, or another target use case. In some embodiments, one or more processors may switch among multiple profiles, update one or more thresholds, or otherwise alter the operating behavior of the system without requiring changes to the overall architecture described herein. Thus, the present disclosure contemplates flexible configuration of the disclosed analysis methods across different clinical and technical deployment environments.

In some embodiments, the various techniques described herein may be used independently or in combination. For example, one embodiment may use the exemplary magnitude-and-symmetry score without visualization or downstream-model integration. Another embodiment may use the broader morphology-score framework together with reduced-lead analysis and serial monitoring. Another embodiment may use HATW-derived labels for downstream AI training without presenting per-lead scores to a clinician. Another embodiment may use one or more visualization, alerting, workflow, or pipeline-integration features without deploying all optional metrics. Accordingly, the disclosed embodiments should not be understood as requiring that all described modules, metrics, models, figures, or workflow branches be used together in every implementation. Rather, the present disclosure encompasses a family of related, processor-executed techniques for analyzing ECG morphology and using the resulting outputs in a variety of diagnostic, training, visualization, and workflow contexts.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, external (e.g., third party) content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B may include the same computing device within the computer system performing both steps or different computing devices within the computer system performing steps A and B.

Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and may be implemented in the form of data that causes functionality to be invoked, e.g., in the form of arguments of a function or API call. To the extent bespoke noun phrases are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

This written description uses examples to disclose the implementations, including the best mode, and to enable any person skilled in the art to practice the implementations, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method for identifying hyperacute T-waves in electrocardiogram (ECG) data, the method comprising:
(a) receiving, by at least one processor, ECG signal data representing cardiac electrical activity of a subject, the ECG signal data comprising waveform data from at least one ECG lead;
(b) identifying, by the at least one processor, at least one T-wave component and at least one QRS complex component within the ECG signal data for the at least one ECG lead;
(c) computing, by the at least one processor, a T-wave magnitude metric for the at least one ECG lead, wherein the T-wave magnitude metric quantifies a size of the T-wave component relative to a size of the QRS complex component;
(d) computing, by the at least one processor, a T-wave symmetry metric for the at least one ECG lead, wherein the T-wave symmetry metric quantifies a temporal distribution of a peak of the T-wave component within a duration of the T-wave component;
(e) generating, by the at least one processor, a hyperacute T-wave (HATW) score for the at least one ECG lead based at least in part on the T-wave magnitude metric and the T-wave symmetry metric; and
(f) producing, by the at least one processor, an output based at least in part on the HATW score, the output being indicative of a presence or absence of a hyperacute T-wave in the at least one ECG lead.

2. The method of claim 1, wherein computing the T-wave magnitude metric comprises computing a ratio of an area under the T-wave component to an amplitude of the QRS complex component, wherein the area under the T-wave component is measured from a J-point to an end of the T-wave, and wherein the amplitude of the QRS complex component is measured as a difference between a maximum value and a minimum value of the QRS complex.

3. The method of claim 1, wherein computing the T-wave symmetry metric comprises computing a ratio of (i) a time interval from a peak of the T-wave component to an end of the T-wave component, to (ii) a time interval from a J-point to the peak of the T-wave component.

4. The method of claim 1, wherein generating the HATW score comprises applying a trained classification model to at least the T-wave magnitude metric and the T-wave symmetry metric, the trained classification model having been trained on expert-annotated ECG data comprising leads labeled as hyperacute or non-hyperacute by human ECG experts.

5. The method of claim 1, wherein:
the ECG signal data comprises waveform data from a plurality of ECG leads;
the method comprises computing the T-wave magnitude metric, the T-wave symmetry metric, and the HATW score for each lead of the plurality of ECG leads; and
producing the output comprises determining that at least two contiguous leads of the plurality of ECG leads each have a HATW score satisfying a predetermined threshold condition.

6. The method of claim 4, wherein the trained classification model comprises a logistic regression model or a feedforward neural network comprising at least two hidden units with logistic activation functions.

7. The method of claim 5, wherein the HATW score is computed using lead-group-specific model parameters, wherein the plurality of ECG leads are divided into at least two lead groups, and wherein each lead group has distinct model parameters optimized for morphological characteristics of that lead group.

8. The method of claim 1, further comprising, prior to computing the T-wave magnitude metric and the T-wave symmetry metric, determining that the at least one ECG lead satisfies one or more eligibility criteria, the one or more eligibility criteria comprising at least one of:
(i) a QRS complex duration not exceeding a predetermined duration threshold;
(ii) a total QRS complex amplitude meeting or exceeding a predetermined QRS amplitude threshold;
(iii) a positive T-wave amplitude meeting or exceeding a predetermined T-wave amplitude threshold;
(iv) a T-wave onset-to-peak duration and a T-wave peak-to-end duration each meeting or exceeding a predetermined minimum T-wave timing threshold; and
(v) the T-wave component being neither inverted nor biphasic according to predetermined morphological criteria.

9. The method of claim 5, wherein the predetermined threshold condition comprises a mean HATW score of the at least two contiguous leads meeting or exceeding a threshold value.

10. The method of claim 9, wherein the threshold value is approximately 0.7.

11. The method of claim 1, wherein generating the HATW score further comprises computing an ST-segment depression metric for the at least one ECG lead, and wherein the HATW score is based at least in part on the T-wave magnitude metric, the T-wave symmetry metric, and the ST-segment depression metric.

12. The method of claim 6, wherein the logistic regression model or feedforward neural network comprises:
a magnitude sub-score computed by applying a first logistic function to at least the area under the T-wave component and the amplitude of the QRS complex component;
a symmetry sub-score computed by applying a second logistic function to at least a T-wave peak-to-end time and a T-wave onset-to-peak time; and
the HATW score computed by applying a third logistic function to at least the magnitude sub-score and the symmetry sub-score.

13. The method of claim 12, wherein the HATW score is computed according to lead-group-specific equations, wherein each equation comprises a logistic function of a weighted combination of a bias term, a scaled magnitude sub-score, and a scaled symmetry sub-score, with lead-group-specific weights and bias values.

14. The method of claim 1, wherein the output is indicative of a likelihood of occlusion myocardial infarction (OMI) in the subject.

15. The method of claim 1, wherein producing the output comprises generating a clinical alert recommending at least one of: emergent cardiac catheterization, emergent reperfusion therapy, or expedited cardiology consultation.

16. The method of claim 1, further comprising constructing, by the at least one processor, at least one median beat representation from the ECG signal data prior to identifying the T-wave component and the QRS complex component.

17. A system for identifying hyperacute T-waves in electrocardiogram (ECG) data, the system comprising:
at least one processor; and
at least one memory storing instructions that, when executed by the at least one processor, cause the system to:
(a) receive ECG signal data representing cardiac electrical activity of a subject, the ECG signal data comprising waveform data from at least one ECG lead;
(b) identify at least one T-wave component and at least one QRS complex component within the ECG signal data for the at least one ECG lead;
(c) compute a T-wave magnitude metric for the at least one ECG lead, wherein the T-wave magnitude metric quantifies a size of the T-wave component relative to a size of the QRS complex component;
(d) compute a T-wave symmetry metric for the at least one ECG lead, wherein the T-wave symmetry metric quantifies a temporal distribution of a peak of the T-wave component within a duration of the T-wave component;
(e) generate a hyperacute T-wave (HATW) score for the at least one ECG lead based at least in part on the T-wave magnitude metric and the T-wave symmetry metric; and
(f) produce an output based at least in part on the HATW score, the output being indicative of a presence or absence of a hyperacute T-wave in the at least one ECG lead.

18. The system of claim 17, wherein:
the ECG signal data comprises waveform data from a plurality of ECG leads;
the instructions further cause the system to compute the T-wave magnitude metric, the T-wave symmetry metric, and the HATW score for each lead of the plurality of ECG leads using lead-group-specific model parameters;
the instructions further cause the system to determine that at least two contiguous leads of the plurality of ECG leads satisfy a predetermined threshold condition based on a mean HATW score of the at least two contiguous leads; and
the output comprises a clinical alert indicative of a likelihood of occlusion myocardial infarction (OMI) and recommending at least one of emergent cardiac catheterization, emergent reperfusion therapy, or expedited cardiology consultation.

19. A non-transitory computer-readable medium storing instructions that, when executed by at least one processor, cause the at least one processor to perform operations comprising:
(a) receiving ECG signal data representing cardiac electrical activity of a subject, the ECG signal data comprising waveform data from at least one ECG lead;
(b) identifying at least one T-wave component and at least one QRS complex component within the ECG signal data for the at least one ECG lead;
(c) computing a T-wave magnitude metric for the at least one ECG lead, wherein the T-wave magnitude metric quantifies a size of the T-wave component relative to a size of the QRS complex component;
(d) computing a T-wave symmetry metric for the at least one ECG lead, wherein the T-wave symmetry metric quantifies a temporal distribution of a peak of the T-wave component within a duration of the T-wave component;
(e) generating a hyperacute T-wave (HATW) score for the at least one ECG lead based at least in part on the T-wave magnitude metric and the T-wave symmetry metric; and
(f) producing an output based at least in part on the HATW score, the output being indicative of a presence or absence of a hyperacute T-wave in the at least one ECG lead.

20. The non-transitory computer-readable medium of claim 19, wherein the operations further comprise:
constructing at least one median beat representation from the ECG signal data prior to identifying the at least one T-wave component and the at least one QRS complex component;
applying a trained classification model to at least the T-wave magnitude metric and the T-wave symmetry metric, the trained classification model having been trained on expert-annotated ECG data comprising leads labeled as hyperacute or non-hyperacute by human ECG experts; and
generating the HATW score further based at least in part on an ST-segment depression metric for the at least one ECG lead.

21. A computer-implemented method for quantifying T-wave morphology in electrocardiogram (ECG) data, the method comprising:
(a) receiving, by at least one processor, ECG signal data comprising waveform data from at least one ECG lead;
(b) extracting, by the at least one processor, from the ECG signal data, at least:
(i) a first metric characterizing a relative size of a T-wave with respect to a QRS complex in the at least one ECG lead, and
(ii) a second metric characterizing a temporal asymmetry or symmetry of the T-wave in the at least one ECG lead;
(c) computing, by the at least one processor, a composite morphology score from at least the first metric and the second metric; and
(d) classifying, by the at least one processor, the T-wave in the at least one ECG lead as hyperacute or non-hyperacute based at least in part on the composite morphology score.
